(19) United States
(12) Patent Application Publication
(10) Pub. No.: US 2011/0028462 A1
(43) Pub. Date: Feb. 3, 2011

(54) Niacin Receptor Agonists, compositions Containing Such Compounds and Methods of Treatment

(76) Inventors: Steven L. Colletti, Princeton Junction, NJ (US); Jason E. Imbriglio, Piscataway, NJ (US); Richard Thomas Beresis, Matawan, NJ (US); Jessica Leslie Frie, Princeton, NJ (US)

Correspondence Address: MERCK, P O BOX 2000, RAHWAY, NJ, 07065-0907, US

(21) Appl. No.: 11/992,069

(22) PCT Filed: Sep. 15, 2006
(86) PCT No.: PCT/US2006/036023

§ 371 Date: Mar. 14, 2008

Related U.S. Application Data
(60) Provisional application No. 60/718,622, filed on Sep. 20, 2005.

(51) Int. Cl. A61K 31/538; A61P 9/10; A61P 3/10; A61P 3/06; A61K 31/381; A61K 31/428; A61K 31/4725; A61K 31/4709; A61K 31/415; A61K 31/403; A61K 31/437

(52) U.S. Cl. 514/230.5; 514/448; 514/367; 514/307; 514/314; 514/406; 514/411; 514/292

(57) ABSTRACT

A method of treating atherosclerosis and related conditions using compounds of formula I: as well as pharmaceutically acceptable salts and solvates is disclosed. The compounds are useful for treating dyslipidemias, and in particular, reducing serum LDL, VLDL and triglycerides, and raising HDL levels.

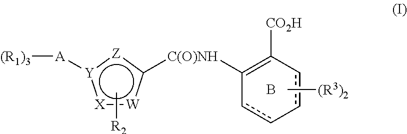

### BACKGROUND OF THE INVENTION

The present invention relates to compounds, compositions and methods of treatment or prevention in a mammal relating to dyslipidemias. Dyslipidemia is a condition wherein serum lipids are abnormal. Elevated cholesterol and low levels of high density lipoprotein (HDL) are associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for cell membranes and essential organic molecules, such as steroids and bile acids, cholesterol in excess is known to contribute to cardiovascular disease. For example, cholesterol is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

The present invention is directed to a method of treating atherosclerosis in a mammalian patient in need of such treatment, comprising administering to the patient an anti-atherosclerotic effective amount of a compound represented by formula I:

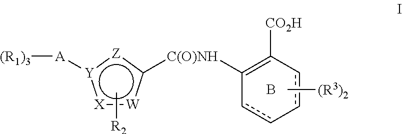

or a pharmaceutically acceptable salt or solvate thereof, wherein:

1-3 of W, X and Z are heteroatoms, and the remaining variable is a carbon atom; Y represents a carbon or nitrogen atom; 0-1 of W, X and Z represent an oxygen or sulfur atom, and the remainder of W, X and Z represent carbon or nitrogen atoms;

A represents a 9-10 membered aryl, an 8-10 membered heteroaryl or a partially aromatic heterocyclic group, said heteroaryl and partially aromatic heterocyclic group containing at least one heteroatom selected from O, S, S(O), S(O)₂ and N, and optionally containing 1 other heteroatom selected from O and S, and optionally containing 1-3 additional N atoms, with up to 5 heteroatoms being present;

each R¹ represents H or is independently selected from the group consisting of:

a) OH, halo, CO₂R^{a}, C(O)NR^{b}R^{c}, NR^{b}R^{c}, CN or S(O)ₚR^{d}; and

b) C₁₋₁₀alkyl, C₂₋₁₀alkenyl, OC₁₋₁₀alkyl or OC₃₋₁₀alkenyl, said groups being optionally substituted with: (1) 1-5 halo groups up to a perhaloalkyl group; (2) 1 oxo group; (3) 1-2 OH groups; (4) 1 phenyl ring, which is optionally substituted as follows: 1-5 halo groups up to perhalo, 1-3 C₁₋₁₀alkyl or alkoxy groups, each being further optionally substituted with 1-5 halo up to perhalo;

R² represents H or is selected from the group consisting of: C₁₋₃alkyl or C₂₋₃alkenyl, said alkyl and alkenyl group being optionally substituted with 1-3 halo atoms, and 1-2 OH, C₁₋₃alkoxy or haloC₁₋₃alkoxy groups;

R^{a} is H or C₁₋₄alkyl, optionally substituted with phenyl, OH, OC₁₋₆alkyl, CO₂H, CO₂C₁₋₆alkyl and 1-3 halo atoms;

R^{b} is H or C₁₋₄alkyl optionally substituted with 1-3 halo atoms and 1 phenyl, OH, and OC₁₋₆alkyl group;

R^{c} is H or is independently selected from: (a) C₁₋₄alkyl, and (b) Aryl or Ar—C₁₋₄alkyl, each optionally substituted with 1-3 halo atoms and 1-3 members selected from the group consisting of: CN, OH, C₁₋₃alkyl and OC₁₋₃ alkyl, said alkyl and alkoxy being further optionally substituted with 1-3 halo atoms;

R^{d} is selected from: (a) C₁₋₄alkyl, (b) Aryl or Ar—C₁₋₄alkyl, each optionally substituted with 1-3 halo atoms and 1-3 members selected from the group consisting of: CN, OH, C₁₋₃alkyl and OC₁₋₃alkyl, said alkyl and alkoxy being further optionally substituted with 1-3 halo atoms;

p is an integer selected from 0, 1 and 2;

and the dotted lines in ring B represent bonds which are either both present or both absent, such that when the bonds are present, ring B is a phenyl ring, and each R³ represents H, halo, methyl or methyl substituted with 1-3 halo atoms;

and when the optional bonds are absent, ring B is a cyclohexene ring and each R³ represents H, halo, C₁₋₃alkyl, Aryl and HAR,

said C₁₋₃alkyl, Aryl and HAR being optionally substituted with 1-3 groups, 0-3 of which are halo, and 0-1 of which are selected from the group consisting of: OH, NH₂, NHC₁₋₃alkyl, N(C₁₋₃alkyl)₂, CN, C₁₋₃alkyl, C₁₋₃alkoxy, haloC₁₋₃alkyl, haloC₁₋₃alkoxy, and Hetcy groups.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means a mono- or bicyclic aromatic ring or ring system containing at least one heteroatom selected from O, S and N, with each ring containing 5 to 6 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzoisothiazolyl, benzimidazolyl, benzofuranyl, benzothiophenyl, benzopyrazolyl, benzotriazolyl, furo(2,3-b)pyridyl, quinolyl, indolyl, isoquinolyl, isoindolyl, quinoxalinyl, quinazolinyl, naphthyridinyl, pteridinyl and the like. Heteroaryl also includes aromatic carbocyclic or heterocyclic groups fused to heterocycles that are non-aromatic or partially aromatic such as indolinyl, dihydrobenzofuranyl, dihydrobenzothiophenyl, dihydrobenzoxazolyl, and aromatic heterocyclic groups fused to cycloalkyl rings. Heteroaryl also includes such groups in charged form, e.g., pyridinium.

“Heterocyclyl” (Hetcy) unless otherwise specified, means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include, but are not limited to, azetidinyl, pyrrolidinyl, piperidinyl, piperazinyl, imidazolidinyl, 2,3-dihydrofuro(2,3-b)pyridyl, tetrahydrofuranyl, benzoxazinyl, 1,4-dioxanyl, tetrahydrohydroquinolinyl, tetrahydroisoquinolinyl, dihydroindolyl, morpholinyl, thiomorpholinyl, tetrahydrothienyl and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils). Heterocyclyl moreover includes such moieties in charged form, e.g., piperidinium.

“Halogen” (Halo) includes fluorine, chlorine, bromine and iodine.

The phrase “in the absence of substantial flushing” refers to the side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity (relative to niacin) is preferably reduced by at least about a third, more preferably by at least half, and most preferably by at least about two thirds. Clearly a one hundred percent reduction in flushing incidence and severity is most preferable, but is not required.

One aspect of the present invention is directed to a method of treating atherosclerosis in a mammalian patient in need of such treatment, comprising administering to the patient an anti-atherosclerotic effective amount of a compound represented by formula I:

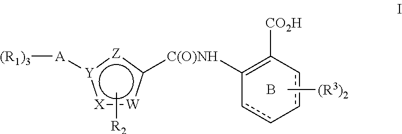

or a pharmaceutically acceptable salt or solvate thereof, wherein:

1-3 of W, X and Z are heteroatoms, and the remaining variable is a carbon atom; Y represents a carbon or nitrogen atom; 0-1 of W, X and Z represent an oxygen or sulfur atom, and the remainder of W, X and Z represent carbon or nitrogen atoms;

A represents a 9-10 membered aryl, an 8-10 membered heteroaryl or a partially aromatic heterocyclic group, said heteroaryl and partially aromatic heterocyclic group containing at least one heteroatom selected from O, S, S(O), S(O)₂ and N, and optionally containing 1 other heteroatom selected from O and S, and optionally containing 1-3 additional N atoms, with up to 5 heteroatoms being present;

each R¹ represents H or is independently selected from the group consisting of:

a) OH, halo, CO₂R^{a}, C(O)NR^{b}R^{c}, NR^{b}R^{c}, CN or S(O)ₚR^{d}; and

b) C₁₋₁₀alkyl, C₂₋₁₀alkenyl, OC₁₋₁₀alkyl or OC₃₋₁₀alkenyl, said groups being optionally substituted with: (1) 1-5 halo groups up to a perhaloalkyl group; (2) 1 oxo group; (3) 1-2 OH groups; (4) 1 phenyl ring, which is optionally substituted as follows: 1-5 halo groups up to perhalo, 1-3 C₁₋₁₀alkyl or alkoxy groups, each being further optionally substituted with 1-5 halo up to perhalo;

R² represents H or is selected from the group consisting of: C₁₋₃alkyl or C₂₋₃alkenyl, said alkyl and alkenyl group being optionally substituted with 1-3 halo atoms, and 1-2 OH, C₁₋₃alkoxy or haloC₁₋₃alkoxy groups;

R^{a} is H or C₁₋₄alkyl, optionally substituted with phenyl, OH, OC₁₋₆alkyl, CO₂H, CO₂C₁₋₆alkyl and 1-3 halo atoms;

R^{b} is H or C₁₋₄alkyl optionally substituted with 1-3 halo atoms and 1 phenyl, OH, and OC₁₋₆alkyl group;

R^{c} is H or is independently selected from: (a) C₁₋₄alkyl, and (b) Aryl or Ar—C₁₋₄alkyl, each optionally substituted with 1-3 halo atoms and 1-3 members selected from the group consisting of: CN, OH, C₁₋₃alkyl and OC₁₋₃ alkyl, said alkyl and alkoxy being further optionally substituted with 1-3 halo atoms;

R^{d} is selected from: (a) C₁₋₄alkyl, (b) Aryl or Ar—C₁₋₄alkyl, each optionally substituted with 1-3 halo atoms and 1-3 members selected from the group consisting of CN, OH, C₁₋₃alkyl and C₁₋₃alkyl, said alkyl and alkoxy being further optionally substituted with 1-3 halo atoms;

p is an integer selected from 0, 1 and 2;

and the dotted lines in ring B represent bonds which are either both present or both absent, such that when the bonds are present, ring B is a phenyl ring, and each R³ represents H, halo, methyl or methyl substituted with 1-3 halo atoms;

and when the optional bonds are absent, ring B is a cyclohexene ring and each R³ represents H, halo, C₁₋₃alkyl, Aryl and HAR,

said C₁₋₃alkyl, Aryl and HAR being optionally substituted with 1-3 groups, 0-3 of which are halo, and 0-1 of which are selected from the group consisting of: OH, NH₂, NHC₁₋₃alkyl, N(C₁₋₃alkyl)₂, CN, C₁₋₃alkyl, C₁₋₃alkoxy, haloC₁₋₃alkyl, haloC₁₋₃alkoxy, and Hetcy groups.

A method of interest within the present invention relates to the treatment of atherosclerosis as described above wherein A represents a member selected from the group consisting of: naphthyl, quinolinyl, isoquinolinyl, quinoxalinyl, benzodioxanyl, benzodioxolanyl, benzodihydrofuranyl and benzothiazolyl. Within this subset of the invention, all other variables are as originally defined with respect to formula I.

Another aspect of the invention that is of interest relates to the method of treating atherosclerosis described above, wherein Z represents a sulfur atom and W, X and Y represent carbon atoms. Within this subset of the invention, all other variables are as originally defined with respect to formula I.

Another aspect of the invention that is of interest relates to the method of treating atherosclerosis described above, wherein W represents a sulfur atom and Z, X and Y represent carbon atoms. Within this subset of the invention, all other variables are as originally defined with respect to formula I.

Another aspect of the invention that is of interest relates to the method of treating atherosclerosis described above, wherein W and Z represent carbon atoms, and X and Y represent nitrogen atoms. Within this subset of the invention, all other variables are as originally defined with respect to formula I.

Another aspect of the invention that is of interest relates to the method of treating atherosclerosis described above, wherein W and X represent carbon atoms, and Y and Z represent nitrogen atoms. Within this subset of the invention, all other variables are as originally defined with respect to formula I.

Another aspect of the invention that is of interest relates to the method of treating atherosclerosis described above, wherein W and Y represent carbon atoms, X represents a sulfur atom and Z represents a nitrogen atom. Within this subset of the invention, all other variables are as originally defined with respect to formula I.

Another aspect of the invention that is of interest relates to the method of treating atherosclerosis described above, wherein W and Y represent carbon atoms, X represents a nitrogen atom and Z represents a sulfur atom. Within this subset of the invention, all other variables are as originally defined with respect to formula I.

Another aspect of the invention that is of interest relates to the method of treating atherosclerosis described above, wherein 1-2 R¹ groups represent H and the remaining R¹ groups are selected from the group consisting of: H, halo, OH, NH₂ and methoxy. Within this subset of the invention, all other variables are as originally defined with respect to formula I.

Another aspect of the invention that is of interest relates to the method of treating atherosclerosis described above, wherein R² represents H or methyl. Within this subset of the invention, all other variables are as originally defined with respect to formula I.

Another aspect of the invention that is of interest relates to the method of treating atherosclerosis described above, wherein R³ represents H. Within this subset of the invention, all other variables are as originally defined with respect to formula I.

More particularly, an aspect of the invention that is of interest relates to a method of treating atherosclerosis as described above, wherein:

A represents a member selected from the group consisting of: naphthyl, quinolinyl, isoquinolinyl, quinoxalinyl, benzodioxanyl, benzodioxolanyl, benzodihydrofuranyl and benzothiazolyl;

Z represents a sulfur atom and W, X and Y represent carbon atoms, or

W represents a sulfur atom and Z, X and Y represent carbon atoms, or

W and Z represent carbon atoms, and X and Y represent nitrogen atoms, or

W and Y represent carbon atoms, X represents a sulfur atom and Z represents a nitrogen atom; or

W and X represent carbon atoms and Y and Z represent nitrogen atoms, or

W and Y represent carbon atoms, X represents a nitrogen atom and Z represents a sulfur atom;

1-2 R¹ groups represent H and the remaining R¹ groups are selected from the group consisting of: H, halo, OH, NH₂ and methoxy;

R² represents H or methyl, and each R³ represents H.

Examples of compounds useful within the present invention are set forth below in Table 1:

TABLE 1

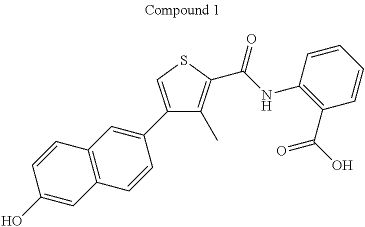

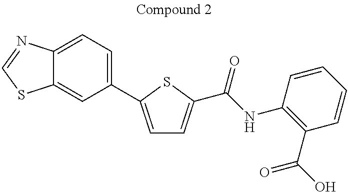

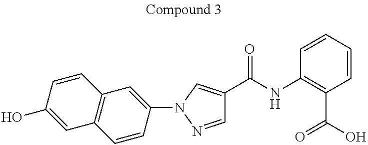

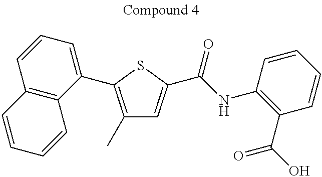

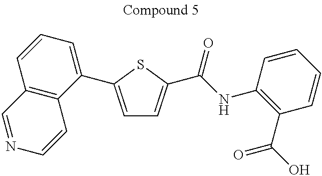

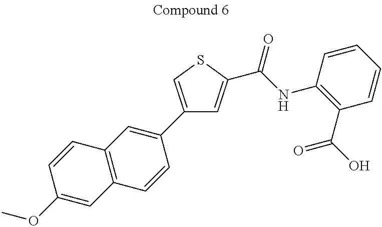

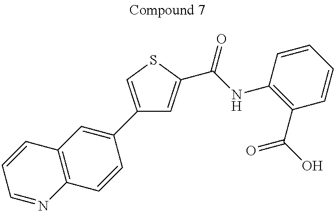

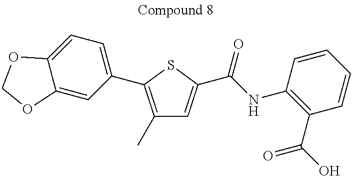

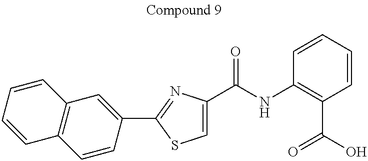

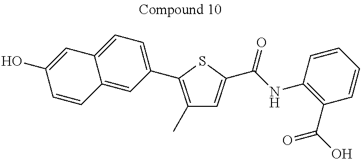

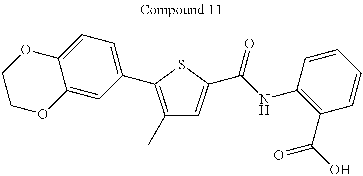

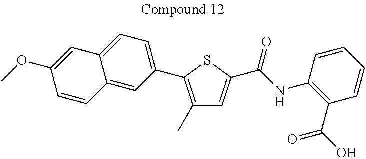

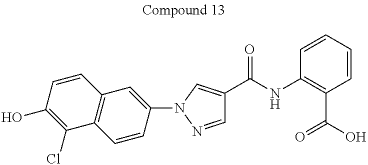

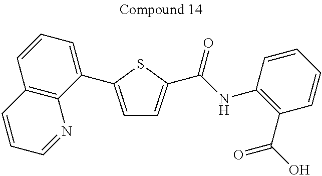

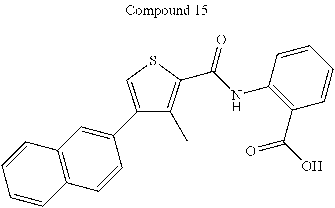

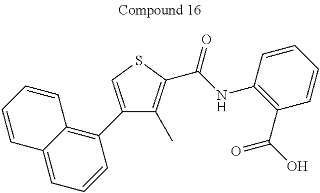

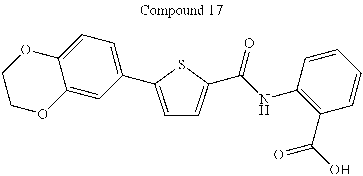

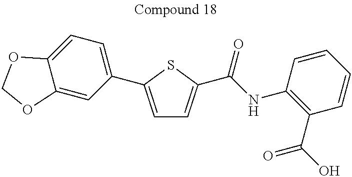

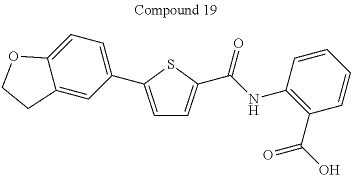

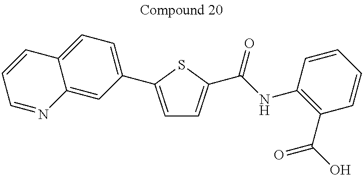

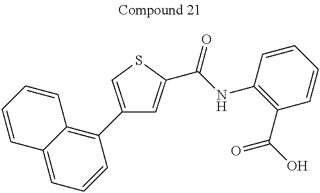

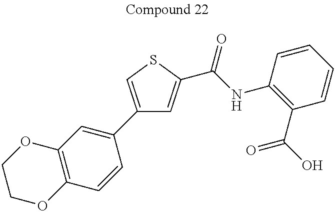

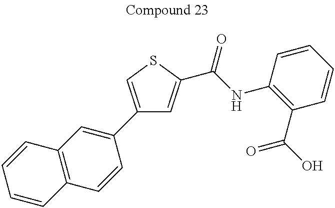

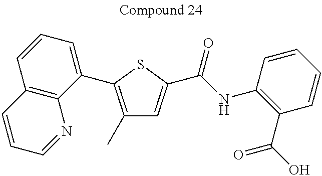

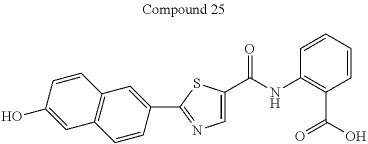

Pharmaceutically acceptable salts and solvates thereof are included as well.

Another aspect of the invention is a method of treating dyslipidemia in a human patient in need of such treatment comprising administering to the patient an anti-dyslipidemic effective amount of a compound represented by formula I or a pharmaceutically acceptable salt or solvate thereof. Within this aspect of the invention, all variables are as originally described with respect to formula I.

Another aspect of the invention is a method of treating diabetes, and in particular, type II or non-insulin dependent diabetes mellitus, in a human patient in need of such treatment comprising administering to the patient an anti-diabetic effective amount of a compound represented by formula I or a pharmaceutically acceptable salt or solvate thereof. Within this aspect of the invention, all variables are as originally described with respect to formula I.

Another aspect of the invention is a method of treating metabolic syndrome in a human patient in need of such treatment comprising administering to the patient a compound represented by formula I or a pharmaceutically acceptable salt or solvate thereof in an amount that is effective to treat metabolic syndrome. Within this aspect of the invention, all variables are as originally described with respect to formula I.

Another aspect of the invention is a method of treating atherosclerosis, dyslipidemia, diabetes, metabolic syndrome or a related condition in a human patient in need of such treatment, comprising administering to the patient a compound of the formula I, or a pharmaceutically acceptable salt or solvate thereof, and a DP receptor antagonist, said combination being administered in an amount that is effective to treat atherosclerosis, dyslipidemia, diabetes or a related condition in the absence of substantial flushing.

More particularly, an aspect of the invention that is of interest relates to a method of treating atherosclerosis, dyslipidemias, diabetes or a related condition in a human patient in need of such treatment, comprising administering to the patient a compound of formula I or a pharmaceutically acceptable salt or solvate thereof and a DP receptor antagonist selected from the group consisting of compounds A through AJ:

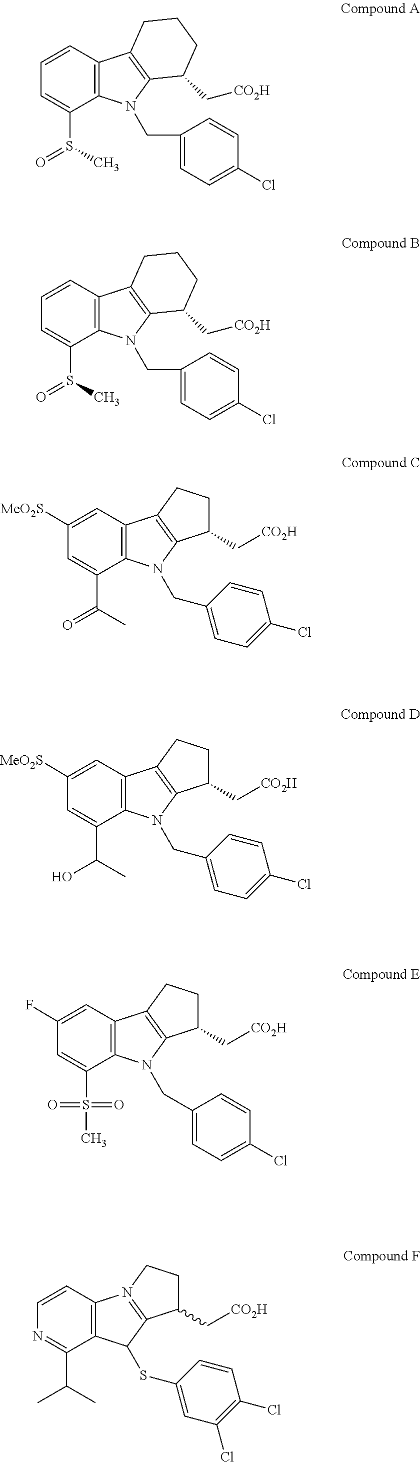

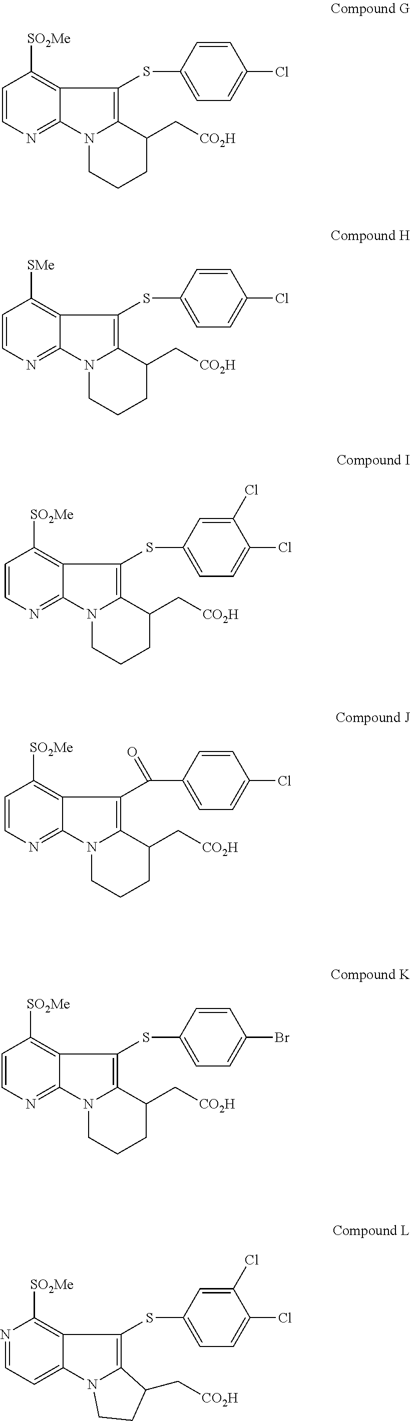

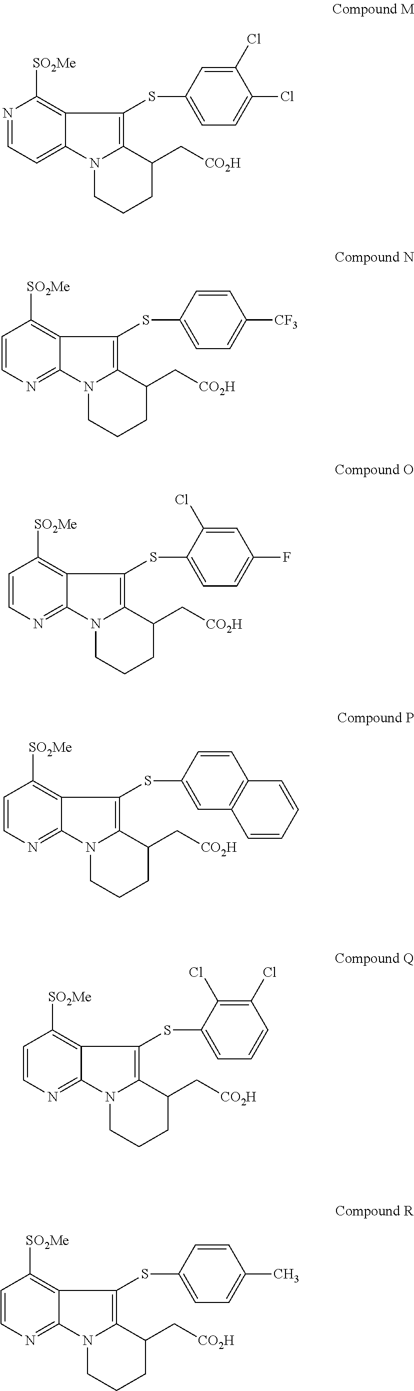

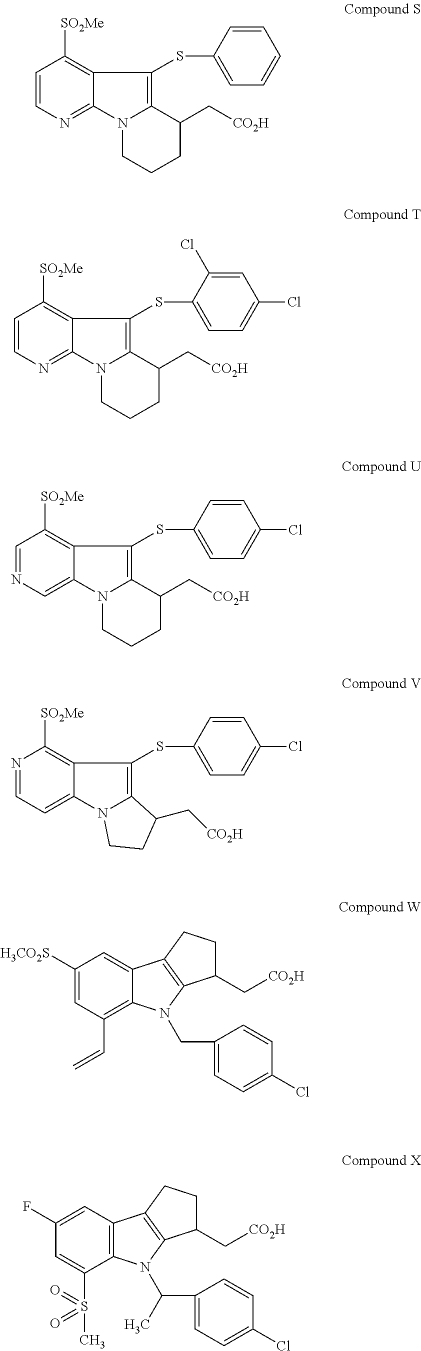

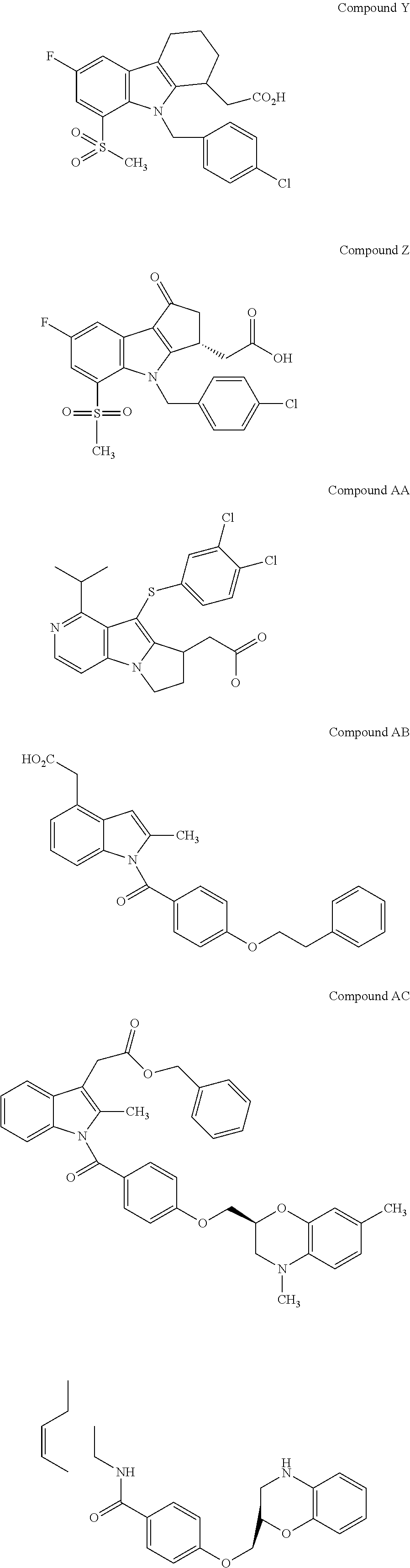

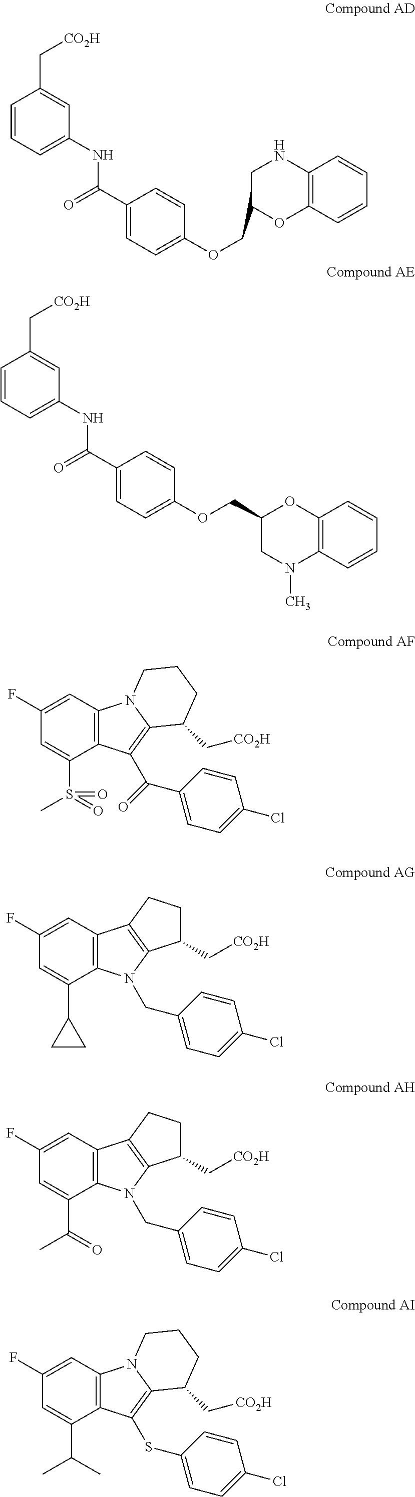

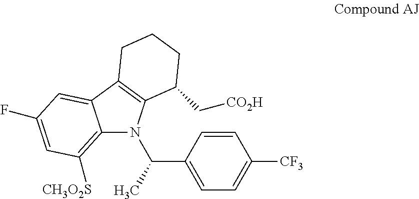

or a pharmaceutically acceptable salt or solvate thereof.

Compounds of formula I may contain asymmetric centers and can thus occur as racemates and racemic mixtures, single enantiomers, diastereomeric mixtures and individual diastereomers. All such isomeric forms are included.

Moreover, chiral compounds possessing one stereocenter of general formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula I to an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I can also be separated directly by chromatographic methods utilizing chiral stationary phases, which methods are well known in the art.

Alternatively, enantiomers of compounds of the general Formula I may be obtained by stereoselective synthesis using optically pure starting materials or reagents.

Some of the compounds described herein exist as tautomers, which have different points of attachment for hydrogen accompanied by one or more double bond shifts. For example, a ketone and its enol form are keto-enol tautomers. Or for example, a 2-hydroxyquinoline can reside in the tautomeric 2-quinolone form. The individual tautomers as well as mixtures thereof are included.

### Dosing Information

The dosages of compounds of formula I or a pharmaceutically acceptable salt or solvate thereof vary within wide limits. The specific dosage regimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage is about 0.1 mg/day to about 1 g/day. Lower dosages can be used initially, and dosages increased to further minimize any untoward effects. It is expected that the compounds described herein will be administered on a daily basis for a length of time appropriate to treat or prevent the medical condition relevant to the patient, including a course of therapy lasting months, years or the life of the patient.

The terms “anti-atherosclerotic effective amount”, “anti-dyslipidemic effective amount”, “anti-diabetic effective amount”, and “amount that is effective to treat metabolic syndrome” refer to the dosages of the compound of formula I that are useful to treat the disease or condition identified. Such dosages may overlap with each other, and typically range from as low as about 0.1 mg to as high as about 2 g, preferably about 1 mg to about 1 g per day, and more preferably about 1 mg to about 200 mg. Dosage adjustment will typically be necessary to take into account the effectiveness of the particular agent selected, the level of therapeutic effect observed, any side effects that are experienced by the patient, and other factors.

### Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy, open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferators activated receptor gamma (PPARγ) agonists including the compounds commonly referred to as glitazones for example pioglitazone and rosiglitazone and, including those compounds included within the structural class known as thiazolidine diones as well as those PPARγ agonists outside the thiazolidine dione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; renin inhibitors, calcium channel blockers such as nifedipine and diltiazem; endothelin antagonists; agents that enhance ABCA1 gene expression; cholesteryl ester transfer protein (CETP) inhibiting compounds, 5-lipoxygenase activating protein (FLAP) inhibiting compounds, 5-lipoxygenase (5-LO) inhibiting compounds, farnesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taking insulin or an oral antidiabetic medication. One example of an oral antidiabetic medication useful herein is metformin.

In the event that these niacin receptor agonists induce some degree of vasodilation, it is understood that the compounds of formula I may be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well. Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower Kᵢ value) than the affinity at the CRTH2 receptor. Any compound that selectively interacts with DP according to these guidelines is deemed “DP selective”.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include the following:

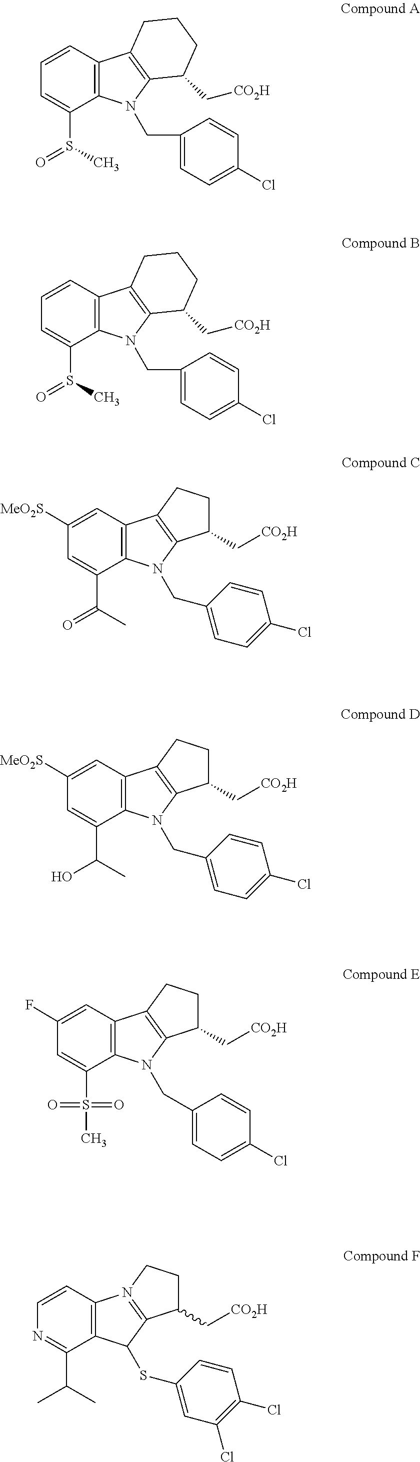

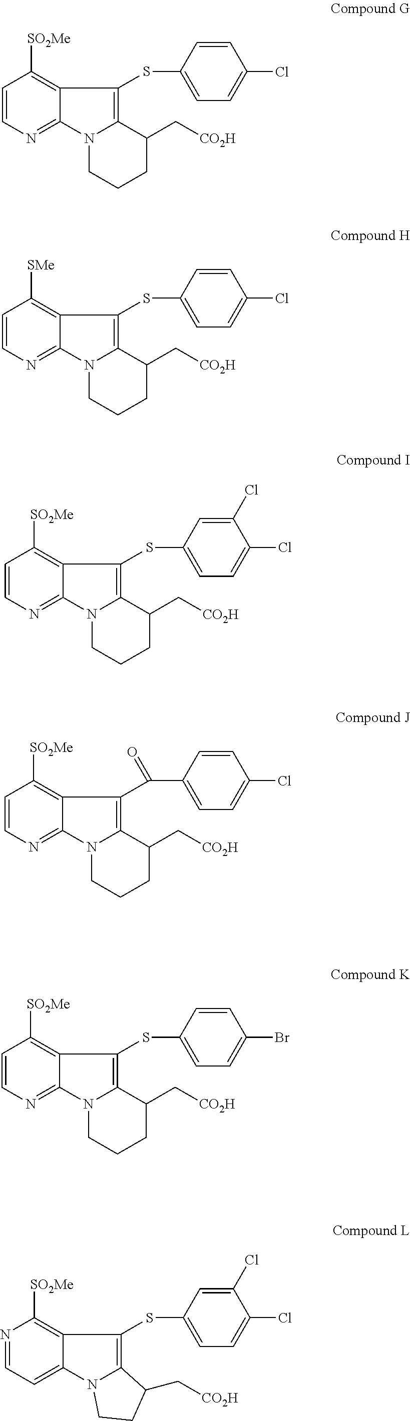

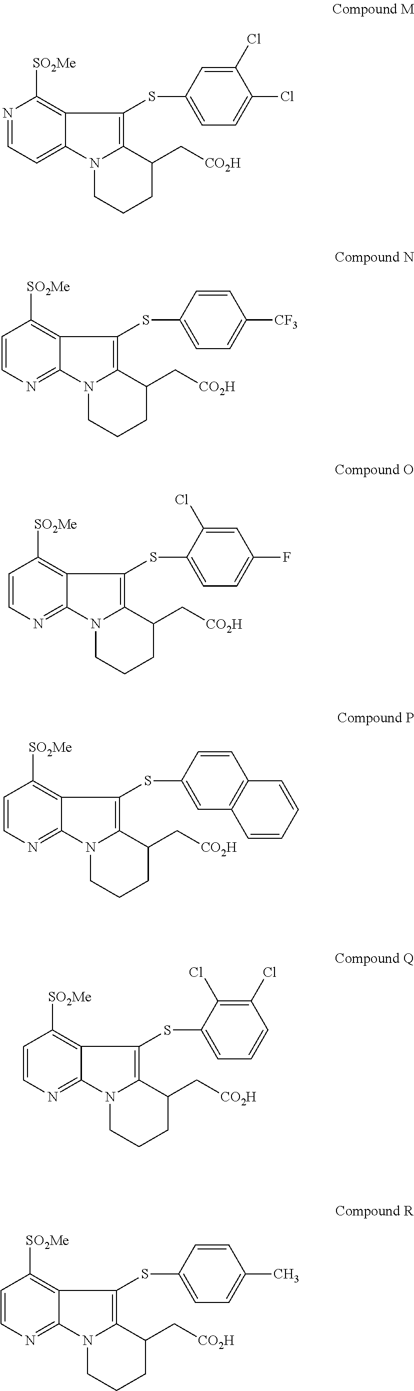

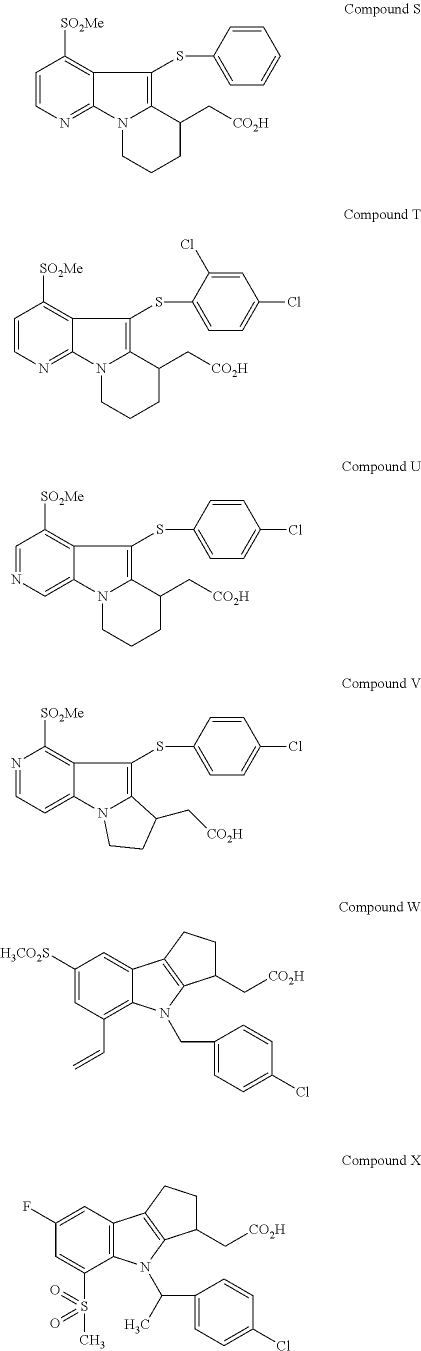

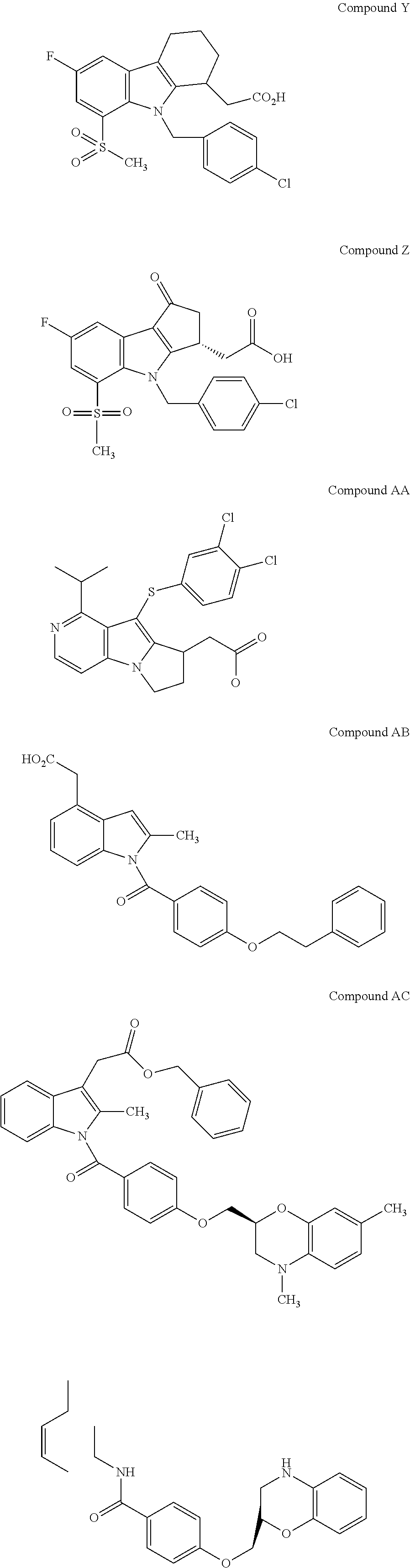

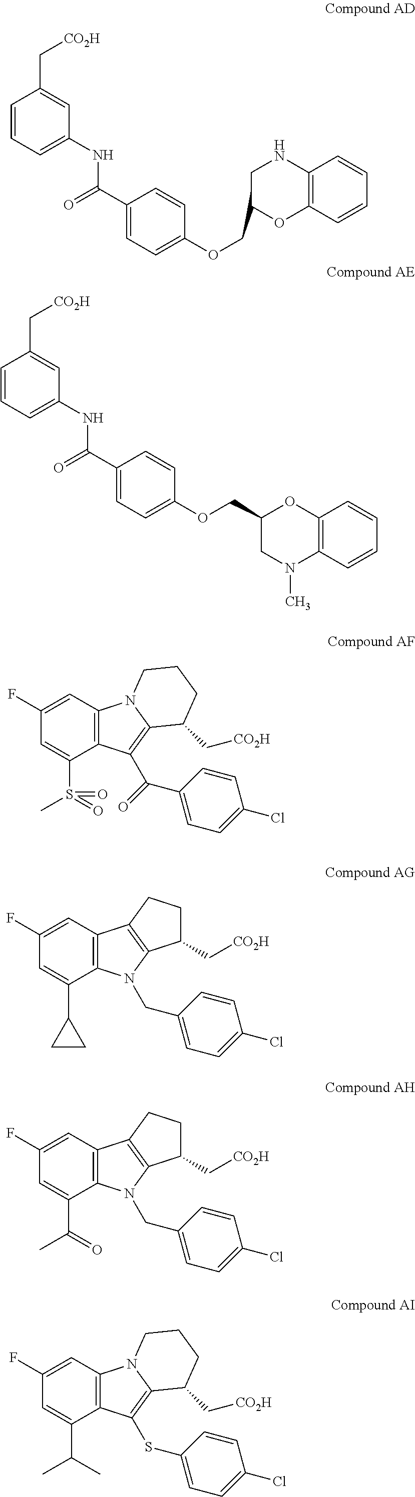

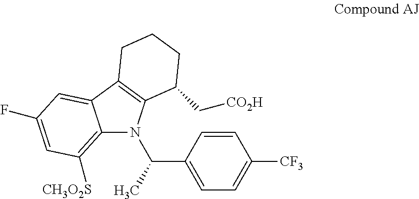

as well as the pharmaceutically acceptable salts and solvates thereof.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

### Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I are useful within the present invention. Examples include the hemihydrate, mono-, di-, tri- and sesquihydrate. The compounds of the invention also include esters that are pharmaceutically acceptable, as well as those that are metabolically labile. Metabolically labile esters include C₁₋₄ alkyl esters, preferably the ethyl ester. Many prodrug strategies are known to those skilled in the art. One such strategy involves engineered amino acid anhydrides possessing pendant nucleophiles, such as lysine, which can cyclize upon themselves, liberating the free acid. Similarly, acetone-ketal diesters, which can break down to acetone, an acid and the active acid, can be used.

The compounds used in the present invention can be administered via any conventional route of administration. The preferred route of administration is oral.

### Pharmaceutical Compositions

The pharmaceutical compositions described and utilized in the methods described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention have anti-hyperlipidemic activity, causing reductions in LDL-C, triglycerides, apolipoprotein a and total cholesterol, and increases in HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, preventing or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, illiliters s, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

### Methods of Synthesis for Compounds of Formula I

Compounds of Formula I have been prepared by the following representative reaction schemes. It is understood that similar reagents, conditions or other synthetic approaches to these structure classes are conceivable to one skilled in the art of organic synthesis. Therefore these reaction schemes should not be construed as limiting the scope of the invention. All substituents are as defined above unless indicated otherwise.

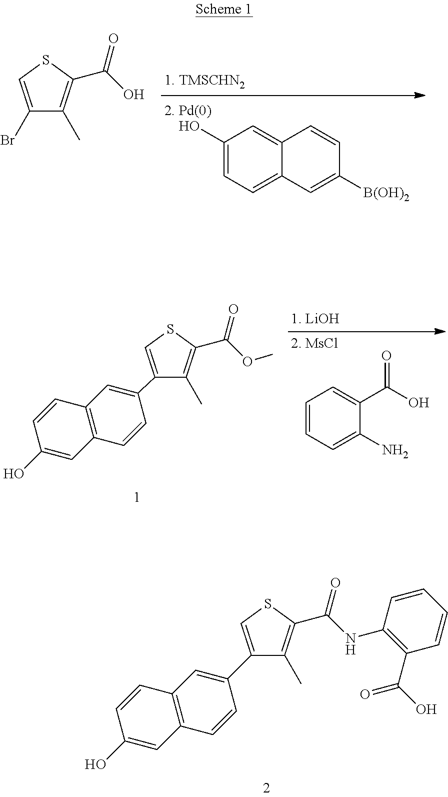

Compounds of Formula I can be prepared as illustrated in Scheme 1 by treatment of a bromothiophene ester with a boronic acid under Suzuki coupling conditions to generate intermediates such as 1. Anthranilic acid can be used directly under mesyl chloride mediated amide coupling conditions with the acid of 1 to generate compounds such as the naphthol anthranilide 2.

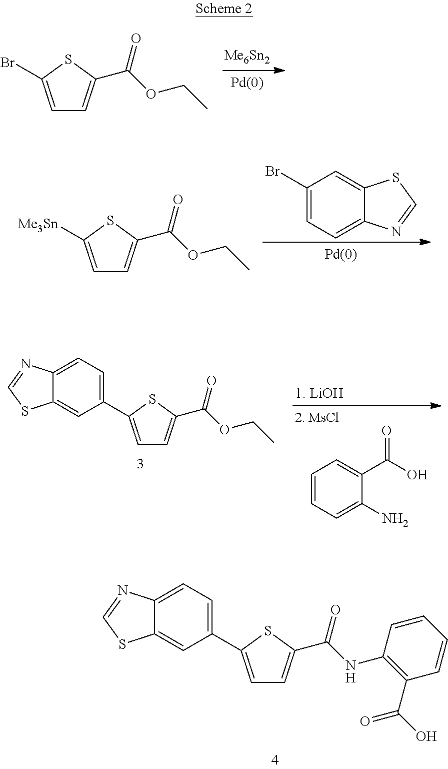

Compounds of Formula I can also be prepared as illustrated in Scheme 2, to access various heterocyclic derivatives. A bromothiophene ester can be converted to its stannane, which in turn can couple with heterocyclic halides, providing intermediates such as 3. The ester 3 can be saponified, and the resulting acid directly coupled with anthranilic acid under mesyl chloride mediated activation conditions to provide compounds such as 4.

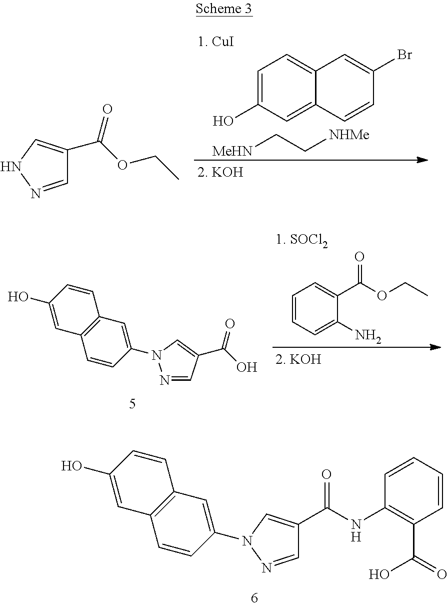

Compounds of Formula I can also be prepared as illustrated in Scheme 3, to access other heterocyclic derivatives, such as pyrazoles. A pyrazole ester can be N-arylated, which in turn can be saponified, providing intermediates such as 5. Activation of 5 to its acid chloride, and the resulting acylation of an anthranilate ester, provides the desired compound such as 6, after saponification.

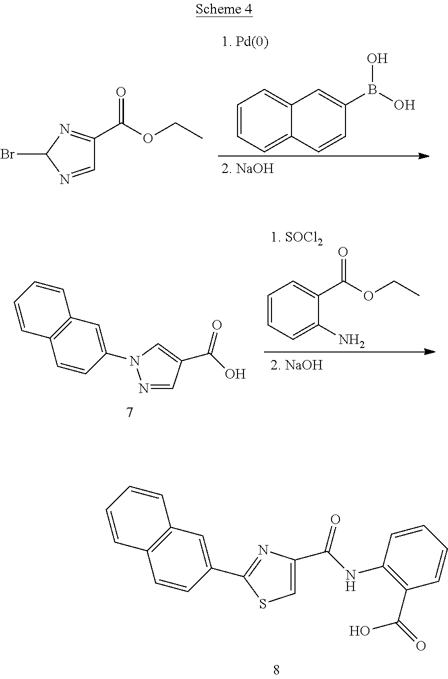

Compounds of Formula I can alternatively be prepared as illustrated in Scheme 4, to access heterocyclic derivatives, such as thiazoles. A bromothiazole ester can be coupled with boronic acids under Suzuki conditions, which in turn can be saponified, providing acid intermediates such as 7. Activation of 7 to its acid chloride, and the resulting acylation of an anthranilate ester, provides the desired compound such as 8, after saponification.

The various organic group transformations and protecting groups utilized herein can be performed by a number of procedures other than those described above. References for other synthetic procedures that can be utilized for the preparation of intermediates or compounds disclosed herein can be found in, for example, M. B. Smith, J. March Advanced Organic Chemistry, 5^{th} Edition, Wiley-Interscience (2001); R. C. Larock Comprehensive Organic Transformations, A Guide to Functional Group Preparations, 2^{nd} Edition, VCH Publishers, Inc. (1999); T. L. Gilchrist Heterocyclic Chemistry, 3^{rd} Edition, Addison Wesley Longman Ltd. (1997); J. A. Joule, K. Mills, G. F. Smith Heterocyclic Chemistry, 3^{rd} Edition, Stanley Thornes Ltd. (1998); G. R. Newkome, W. W. Paudler Contemporary Heterocyclic Chemistry, John Wiley and Sons (1982); or Wuts, P. G. M.; Greene, T. W.; Protective Groups in Organic Synthesis, 3^{rd} Edition, John Wiley and Sons, (1999), all six incorporated herein by reference in their entirety.

### REPRESENTATIVE EXAMPLES

The following examples are provided to more fully illustrate the present invention, and shall not be construed as limiting the scope in any manner. Unless stated otherwise:

(i) all operations were carried out at room or ambient temperature, that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) yields, if given, are for illustration only;

(v) the structure of all final compounds was assured by at least one of the following techniques: MS or proton nuclear magnetic resonance (¹H NMR) spectrometry, and the purity was assured by at least one of the following techniques: TLC or HPLC;

(vi) ¹H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. Broad; etc.;

(vii) MS data were recorded on a Waters Micromass unit, interfaced with a Hewlett-Packard (Agilent 1100) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid-acetonitrile, A=0.1% formic acid-water), Waters Xterra C18-3.5 um-50×3.0 mmID and diode array detection;

(viii) the purification of compounds by preparative reverse phase HPLC (RPHPLC) was conducted on either a Waters Symmetry Prep C18-5 um-30×100 mmID, or a Waters Atlantis Prep dC18-5 um-20×100 mmID; 20 mL/min, 10-100% B linear gradient over 15 min (B=0.05% TFA-acetonitrile, A=0.05% TFA-water), and diode array detection;

(ix) automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA);

(x) the purification of compounds by preparative thin layer chromatography (PTLC) was conducted on 20×20 cm glass prep plates coated with silica gel, commercially available from Analtech;

(xi) column chromatography was carried out on a Biotage cartridge system;

(xii) chemical symbols have their usual meanings; the following abbreviations have also been used v (volume), w (weight), b.p. (boiling point), m.p. (melting point), L (litre(s)), mL (illiliters), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar);

(xiii) definitions of acronyms are as follows:
THF is tetrahydrofuran;
DMF is dimethylformamide;
Pd(PPh₃)₄ is tetrakis triphenylphosphine palladium (0);
TFA is trifluoroacetic acid;
DMSO is dimethyl sulfoxide

### Example 1

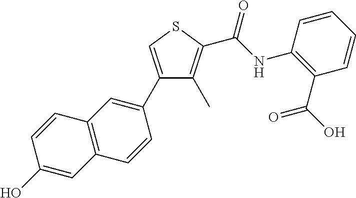

A solution of 4-bromo-3-methylthiophene carboxylic acid (1 g) in methanol was treated with a stock solution of trimethylsilyl diazomethane (2M hexanes) in excess. Upon full consumption of the carboxylic acid, the reaction mixture was quenched with acetic acid and concentrated in vacuo. This bromo methyl ester intermediate (100 mg, 0.43 mmol) was diluted into (0.1M, 1:1) ethanol-dioxane co-solvent, and combined with 1M aqueous sodium bicarbonate (0.85 mL, 0.85 mmol), lithium chloride (36 mg, 0.85 mmol), 6-hydroxy-2-naphthyl boronic acid (160 mg, 0.85 mmol), and catalytic Pd(Ph₃P)₄. The reaction mixture was heated at reflux overnight, cooled, concentrated in vacuo, partitioned between water and methylene chloride, the organic phase concentrated, and the residue purified by preparative RPHPLC. The methyl ester was saponified at room temperature using excess 1M aqueous lithium hydroxide in (3:1:1) THF-methanol-water. The reaction mixture was concentrated in vacuo to remove volatiles, acidified with 1N aqueous HCl, and partitioned with water and 30% isopropanol-chloroform. The organic phase was concentrated in vacuo, and the dried solid (79 mg, 0.28 mmol) was diluted into methylene chloride (0.1 M), treated with triethylamine (0.12 mL, 0.83 mmol), methanesulfonyl chloride (0.027 mL, 0.34 mmol), and anthranilic acid (38 mg, 0.28 mmol). The reaction mixture was maintained at room temperature overnight, concentrated in vacuo, the solid acid (as naphthol sulfonate) treated with excess 1M aqueous lithium hydroxide in (3:1:1) THF-methanol-water, again concentrated in vacuo to remove volatiles, acidified with 1N aqueous HCl to pH7, and purified by preparative RPHPLC. ¹H NMR (DMSO-d₆, 500 MHz) δ 9.8 (s, 1H), 8.6 (d, 1H), 8.1 (d, 1H), 7.8 (m, 4H), 7.6 (t, 1H), 7.4 (d, 1H), 7.2 (m, 3H), 2.5 (s, 3H); LCMS m/z 404 (M+1).

### Example 2

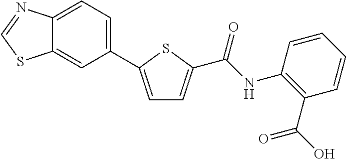

A mixture of ethyl 5-bromothiophene carboxylate (100 mg, 0.43 mmol), hexamethylditin (209 mg, 0.64 mmol), and catalytic Pd(Ph₃P)₄ in 2 mL of THF was heated at 70° C. overnight, cooled, concentrated in vacuo, and purified by preparative TLC (pre-treated plates with 5% triethylamine-hexane; developed plates in 30% ethyl acetate-hexane). This stannane intermediate (100 mg, 0.31 mmol) was combined with 6-bromo-1,3-benzothiazole (150 mg, 0.71 mmol), catalytic Pd(Ph₃P)₄ and diluted in toluene (0.1 M). The reaction mixture was heated at 100° C. overnight, cooled, filtered over celite, concentrated in vacuo, and purified by preparative reverse phase HPLC. The ethyl ester was saponified at room temperature using excess 1M aqueous lithium hydroxide in (3:1:1) THF-methanol-water. The reaction mixture was concentrated in vacuo to remove volatiles, acidified with 1N aqueous HCl, and partitioned with water and 30% isopropanol-chloroform. The organic phase was concentrated in vacuo, and the dried solid (6 mg, 0.023 mmol) was diluted into methylene chloride (0.1 M), treated with triethylamine (0.016 mL, 0.12 mmol), methanesulfonyl chloride (0.003 mL, 0.03 mmol), and anthranilic acid (3 mg, 0.023 mmol). The reaction mixture was maintained at room temperature overnight, concentrated in vacuo, and purified by preparative RPHPLC. ¹H NMR (DMSO-d₆, 500 MHz) δ 12.2 (s, 1H), 9.4 (s, 1H), 8.6 (s, 1H), 8.5 (d, 1H), 8.15 (d, 1H), 8.1 (d, 1H), 7.9 (d, 1H), 7.8 (m, 2H), 7.7 (t, 1H), 7.2 (t, 1H); LCMS m/z 380 (M⁺).

### Examples 3-21

The following compounds were prepared under conditions similar to those described in Examples 1 and 2 above, and illustrated in Schemes 1 and 2.

| EXAMPLE | | LCMS (m/z) |
|---|---|---|
| 3 | 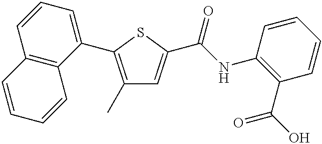 | 387 (M⁺) |
| 4 | 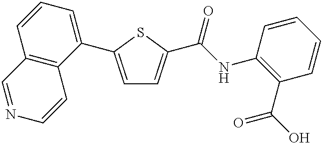 | 375 (M + 1) |
| 5 | 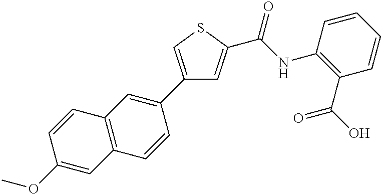 | 403 (M⁺) |
| 6 | 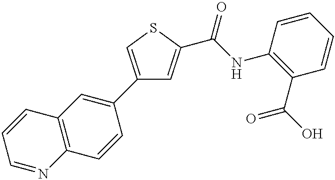 | 374 (M⁺) |
| 7 | 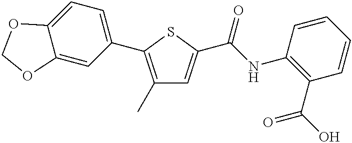 | 382 (M + 1) |
| 8 | 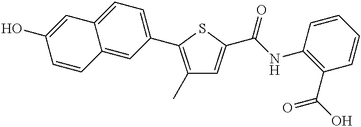 | 404 (M + 1) |
| 9 | 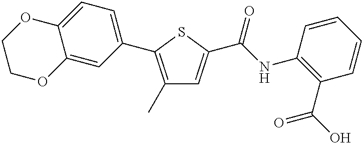 | 396 (M + 1) |
| 10 | 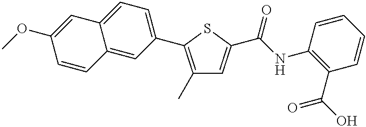 | 418 (M + 1) |
| 11 | 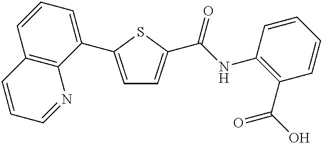 | 375 (M + 1) |
| 12 | 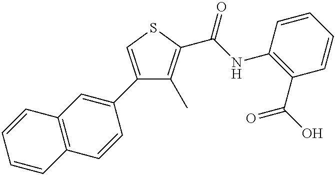 | 388 (M + 1) |
| 13 | 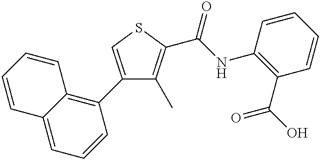 | 388 (M + 1) |
| 14 | 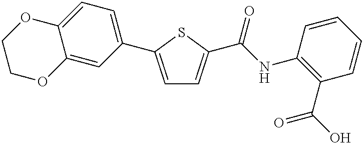 | 381 (M⁺) |
| 15 | 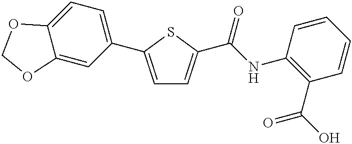 | — |
| 16 | 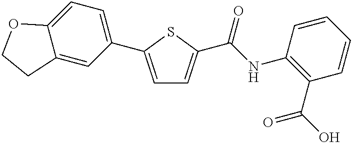 | 365 (M⁺) |
| 17 | 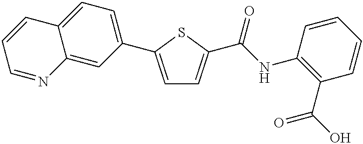 | 374 (M⁺) |
| 18 | 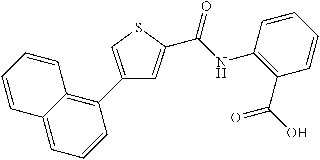 | 373 (M⁺) |
| 19 | 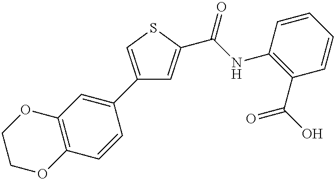 | 381 (M⁺) |
| 20 | 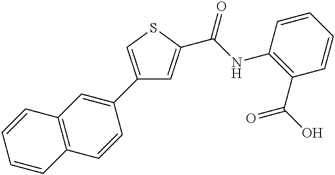 | 373 (M⁺) |
| 21 | 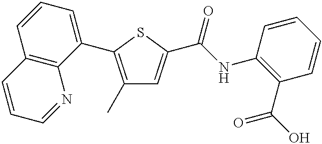 | 389 (M + 1) |

NMR data for selected Examples:

### Example 3

¹H NMR (DMSO-d₆, 500 MHz) δ 12.2 (s, 1H), 8.6 (d, 1H), 8.15 (m, 3H), 7.8 (s, 1H), 7.6 (m, 6H), 7.2 (t, 1H), 2.0 (s, 3H).

### Example 4

¹H NMR (DMSO-d₆, 500 MHz) δ 12.2 (s, 1H), 9.5 (s, 1H), 8.6 (m, 2H), 8.3 (d, 1H), 8.15 (m, 3H), 7.9 (m, 3H), 7.7 (t, 1H), 7.6 (d, 1H), 7.2 (t, 1H).

### Example 5

¹H NMR (DMSO-d₆, 500 MHz) δ 12.2 (s, 1H), 8.6 (d, 1H), 8.3 (d, 2H), 8.2 (s, 1H), 8.1 (d, 1H), 7.9 (m, 4H), 7.6 (m, 1H), 7.4 (s, 1H) 7.2 (m, 1H), 4.0 (s, 3H).

### Example 6 ¹H NMR (DMSO-d₆, 500 MHz) δ 12.2 (s, 1H), 9.0 (d, 1H), 8.6 (t, 2H), 8.4 (s, 2H), 8.3 (s, 1H), 8.2 (d, 1H), 8.15 (d, 1H), 8.0 (d, 1H), 7.6 (m, 2H), 7.2 (t, 1H).

### Example 7

¹H NMR (DMSO-d₆, 500 MHz) δ 12.1 (s, 1H), 8.6 (d, 1H), 8.0 (s, 1H), 7.65 (t, 1H), 7.55 (s, 1H), 7.2 (t, 1H), 7.1 (s, 1H), 7.0 (m, 2H), 6.1 (s, 2H), 2.3 (s, 3H).

### Example 8

¹H NMR (DMSO-d₆, 500 MHz) δ 9.9 (s, 1H), 8.6 (d, 1H), 8.1 (d, 1H), 7.95 (s, 1H), 7.9 (d, 1H), 7.8 (d, 1H), 7.65 (s, 1H), 7.55 (d, 1H), 7.2 (m, 3H), 2.4 (s, 3H).

### Example 9

¹H NMR (DMSO-d₆, 500 MHz) δ 12.1 (s, 1H), 8.6 (d, 1H), 8.1 (s, 1H), 7.85 (t, 1H), 7.75 (s, 1H), 7.2 (t, 1H), 7.0 (m, 3H), 4.3 (s, 4H), 2.3 (s, 3H).

### Example 10

¹H NMR (DMSO-d₆, 500 MHz) δ 12.2 (s, 1H), 8.6 (d, 1H), 8.15 (m, 2H), 7.95 (m, 2H), 7.65 (m, 3H), 7.4 (s, 1H), 7.2 (m, 2H), 3.9 (s, 3H), 2.4 (s, 3H).

### Example 11

¹H NMR (DMSO-d₆, 500 MHz) δ 12.2 (s, 1H), 9.1 (s, 1H), 8.6 (d, 1H), 8.5 (d, 1H), 8.4 (d, 1H), 8.0 (m, 3H), 7.7 (m, 4H), 7.2 (t, 1H).

### Example 12

¹H NMR (DMSO-d₆, 500 MHz) δ 11.9 (s, 1H), 8.6 (d, 1H), 8.0 (m, 5H), 7.9 (s, 1H), 7.65 (t, 1H), 7.6 (m, 3H), 7.2 (t, 1H), 2.5 (s, 3H).

### Example 13

¹H NMR (DMSO-d₆, 500 MHz) δ 12.0 (s, 1H), 8.65 (d, 1H), 8.15 (m, 3H), 7.8 (s, 1H), 7.65-7.4 (m, 6H), 7.2 (t, 1H), 2.2 (s, 3H).

### Example 14

¹H NMR (DMSO-d₆, 500 MHz) δ 8.6 (d, 1H), 8.0 (d, 1H), 7.7 (d, 1H), 7.65 (t, 1H), 7.55 (d, 1H), 7.2 (m, 3H), 6.95 (d, 1H), 4.3 (s, 2H).

### Example 15

¹H NMR (DMSO-d₆, 500 MHz) δ 8.6 (d, 1H), 8.0 (d, 1H), 7.7 (d, 1H), 7.5 (m, 2H), 7.4 (s, 1H), 7.2 (d, 1H), 7.1 (t, 1H), 7.0 (d, 1H), 6.1 (s, 2H).

### Example 16

¹H NMR (DMSO-d₆, 500 MHz) δ 12.2 (s, 1H), 8.6 (d, 1H), 8.0 (d, 1H), 7.7 (m, 3H), 7.5 (m, 2H), 7.2 (t, 1H), 6.8 (d, 1H), 4.6 (t, 2H), 3.25 (t, 2H).

### Example 17

¹H NMR (DMSO-d₆, 500 MHz) δ 12.2 (s, 1H), 9.0 (d, 1H), 8.6 (t, 2H), 8.5 (s, 1H), 8.2 (d, 1H), 8.15 (d, 1H), 8.1 (d, 1H), 7.9 (d, 1H) 7.8 (d, 1H), 7.7 (t, 2H), 7.2 (t, 1H).

### Example 18

¹H NMR (DMSO-d₆, 500 MHz) δ 12.3 (s, 1H), 8.6 (d, 1H), 8.1 (s, 1H), 8.0 (m, 4H), 7.9 (s, 1H), 7.62 (t, 1H), 7.6 (m, 4H), 7.2 (t, 1H).

### Example 19

¹H NMR (DMSO-d₆, 500 MHz) δ 12.2 (s, 1H), 8.5 (d, 1H), 8.15 (s, 1H), 8.0 (m, 2H), 7.6 (t, 1H), 7.2 (m, 3H), 6.9 (d, 1H), 4.3 (m, 4H).

### Example 20

¹H NMR (DMSO-d₆, 500 MHz) δ 12.2 (s, 1H), 8.6 (d, 1H), 8.4 (s, 1H), 8.3 (d, 2H), 8.1 (d, 1H), 8.0 (d, 1H), 7.9 (m, 4H), 7.7 (t, 1H) 7.5 (m, 2H), 7.2 (t, 1H).

### Example 21

¹H NMR (DMSO-d₆, 500 MHz) δ 12.1 (s, 1H), 9.0 (s, 1H), 8.6 (d, 1H), 8.5 (d, 1H), 8.15 (d, 1H), 8.1 (d, 1H), 7.8 (d, 1H), 7.6 (m, 4H), 7.2 (t, 1H), 2.2 (s, 3H).

### Example 22

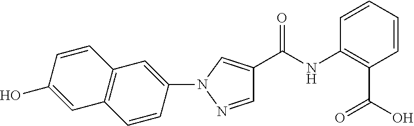

To a solution of ethyl 4-pyrazolecarboxylate (100 mg, 0.71 mmol) in anhydrous toluene (5 mL), under nitrogen, was added 6-bromo-2-naphthol (197 mg, 0.86 mmol), potassium carbonate (206 mg, 1.49 mmol), dimethylethylenediamine (12.5 mg, 0.14 mmol), and copper iodide (6.7 mg, 0.03 mmol). The reaction was then stirred at 110° C. for 24 h. Upon completion, the reaction was filtered and concentrated in vacuo. To the filtrate was added 50 mL of EtOAc and the organic layer was washed with 1M HCl, brine, and dried over Na₂SO₄ and concentrated in vacuo. To this naphthol pyrazole ester (134 mg, 0.5 mmol) in THF (2 mL), MeOH (1 mL), and water (2 mL), was added potassium hydroxide (160 mg, 2.85 mmol). The reaction mixture was stirred at 35° C. for 12 h, at which time the reaction was concentrated in vacuo. Water (5 mL) was added to the residue, and the aqueous layer was acidified with concentrated HCl to pH 2. The acidic solution was extracted with ethyl acetate and the organic layer was separated, dried over Na₂SO₄, and concentrated in vacuo. To this acid intermediate (76 mg, 0.3 mmol), was added toluene (5 mL), followed by thionyl chloride (342 mg, 3 mmol). After stirring the reaction mixture at 90° C. for 2 h, the solution was concentrated in vacuo. To this resultant acid chloride was added methylene chloride (5 mL) followed by ethyl anthranilate (239 mg, 1.45 mmol), and the reaction was allowed to stir at room temperature for 5 h. Upon completion, the mixture was washed with aqueous 1M HCl, saturated sodium bicarbonate, brine, and dried over Na₂SO₄ and concentrated in vacuo. To the ester intermediate (20 mg, 0.05 mmol) in THF (2 mL), MeOH (1 mL), and water (2 mL), was added potassium hydroxide (160 mg, 2.85 mmol), and the reaction mixture was stirred at 50° C. for 12 h. Following reaction completion, the mixture was concentrated in vacuo, followed by the addition of water (5 mL), and the aqueous layer was acidified with concentrated HCl to pH 2. The acidic solution was extracted with ethyl acetate and the organic layer was separated, dried over Na₂SO₄, and filtered. The filtrate was concentrated in vacuo and purified by reverse phase HPLC (Gilson) to give the desired compound. ¹H NMR (DMSO-d₆, 500 MHz) δ 11.89 (s, 1H), 9.93 (s, 1H), 9.11 (s, 1H), 8.62 (d, 1H), 8.34 (s, 1H), 8.20 (s, 1H), 8.06 (d, 1H), 7.99 (d, 1H), 7.89 (s, 2H), 7.67 (m, 1H), 7.20 (m, 2H); LCMS m/z 374 (M+1).

### Example 23

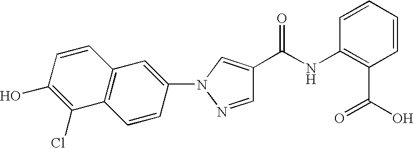

6-Benzyloxy-2-bromo-5-chloro-napthalene (330 mg) and ethyl 4-pyrazolecarboxylate (140 mg) were dissolved in 5 mL degassed toluene. To this was added potassium carbonate (290 mg), CuI (19 mg), and N,N-dimethyl-ethylenediamine (34 mg) and the resulting mixture was stirred at 110° C. for 15 h. Following cooling, filtration and evaporation, preparatory thin layer chromatography (ethyl acetate/dichloromethane eluent) yielded the desired N-napthyl pyrazole intermediate. This compound (200 mg) was subjected to hydrolysis conditions (3 mL THF, 3 mL MeOH, 5 mL 1N aqueous LiOH) for 3 h at room temperature, at which time the desired carboxylic acid precipitated from solution, and was collected by filtration. This intermediate (60 mg) was dissolved in 5 mL of dichloromethane and cooled to 0° C. Then, oxayl chloride (2M, 0.5 mL) and DMF (0.03 mL) were added, and the resulting reaction mixture was heated to 40° C. for 30 minutes. Solvent was then evaporated, and the resulting acid chloride residue was diluted into tetrahydrofuran (5 mL) and triethylamine (0.12 mL), and benzyl anthranilate (93 mg) was added. This reaction mixture was stirred at room temperature for 15 h before being evaporated under reduced pressure. The desired product was purified by trituration with methanol. This dibenzyl-protected intermediate (25 mg) was then dissolved in a mixture of methanol and dichloromethane (10 mL) and palladium hydroxide (10 mg) was added, and the resulting reaction mixture was stirred vigorously under a hydrogen atmosphere for 4 h. Filtration and reverse phase HPLC purification gave the desired product. ¹H NMR (600 MHz, DMSO-d₆) δ 10.61 (s, 1H), 9.13 (s, 1H), 8.57 (d, 1H), 8.44 (d, 1H), 8.18-8.13 (m, 2H), 8.02 (dd, 1H), 7.86 (d, 1H), 7.63 (t, 1H), 7.36 (d, 1H), 7.17 (dd, 1H); LCMS m/z 430 (M+Na).

### Example 24

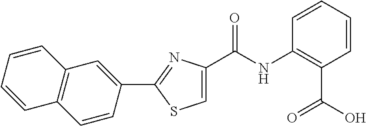

To a solution ethyl 2-bromothiazole-4-carboxylate (100 mg, 0.43 mmol) in anhydrous dioxane (1 mL), under nitrogen, was added 2-naphthaleneboronic acid (147 mg, 0.86 mmol), triethylamine (130 mg, 1.28 mmol), and Pd(Ph₃P)₄ (24 mg, 0.02 mmol). The reaction mixture was heated in a microwave reactor at 100° C. (100 W) for 10 min, partitioned with aqueous 1M NaOH, washed with brine, and the organic phase was dried over anhydrous sodium sulfate, and concentrated in vacuo. To this naphthyl thiazole ester (81 mg, 0.3 mmol) in THF (2 mL), MeOH (1 mL), and water (2 mL), was added aqueous 1M sodium hydroxide (120 mg, 3.0 mmol). The reaction mixture was stirred at 35° C. for 12 h, at which time the mixture was concentrated in vacuo. Water (5 mL) was added to the residue, and the aqueous layer was acidified with concentrated HCl to pH 2. The acidic solution was extracted with ethyl acetate, and the organic layer was separated, dried over Na₂SO₄, and concentrated in vacuo. To this naphthyl thiazole acid intermediate (47 mg, 0.18 mmol), was added toluene (5 mL), followed by thionyl chloride (149 mg, 1.26 mmol). After stirring the reaction mixture at 90° C. for 2 h, the solution was concentrated in vacuo. To this acid chloride intermediate was added methylene chloride (5 mL) followed by ethyl anthranilate (104 mg, 0.63 mmol), and the reaction mixture was allowed to stir at room temperature for 5 h. Upon reaction completion, the mixture was washed with a solution of 1M HCl, saturated sodium bicarbonate, brine, and dried over Na₂SO₄ and concentrated in vacuo. To the naphthyl thiazole anthranilide ester (35 mg, 0.09 mmol) in THF (2 mL), MeOH (1 mL), and water (2 mL), was added aqueous 1M sodium hydroxide (36 mg, 0.9 mmol), and the reaction mixture was stirred at 50° C. for 12 h. Following reaction completion, the mixture was concentrated in vacuo, followed by the addition of water (5 mL), and the) aqueous layer was acidified with concentrated HCl to pH 2. The acidic solution was extracted with ethyl acetate, and the organic layer was separated, dried over Na₂SO₄, and filtered. The filtrate was concentrated in vacuo and purified by reverse phase HPLC (Gilson) to give the desired compound. ¹H NMR (DMSO-d₆, 500 MHz) δ 8.88 (d, 1H), 8.66 (s, 1H), 8.61 (s, 1H), 8.31 (d, 1H), 8.12 (m, 4H), 7.68 (m, 2H), 7.27 (m, 2H); LCMS m/z 375 (M+1).

### Example 25

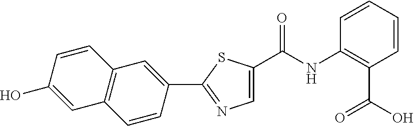

To a solution of ethyl 2-bromothiazole-5-carboxylate (1 g, 4.2 mmol) in anhydrous toluene (10 mL), under nitrogen, was added 6-methoxy-2-naphthaleneboronic acid (1.71 g, 8.5 mmol), potassium carbonate (1.76 g, 12.7 mmol) in water (3 mL), and Pd(Ph₃P)₄ (97 mg, 0.084 mmol). The reaction mixture was heated in a pressure tube for 12 hours. Upon completion, the reaction was washed with brine, and the organic phase was dried over anhydrous sodium sulfate, and concentrated in vacuo. To this naphthyl thiazole ester (300 mg, 0.96 mmol) in CH₂Cl₂(5 mL), at 0° C. was added boron tribromide (1M solution, 9.6 mL, 9.6 mmol). The reaction mixture was stirred at room temperature for 2 h. Upon completion, water (5 mL) was added to quench the reaction and the organic layer was separated, dried over Na₂SO₄, and concentrated in vacuo. To this naphthol thiazole acid intermediate (230 mg, 0.85 mmol), was added toluene (5 mL), followed by thionyl chloride (1 g, 8.5 mmol). After stirring the reaction mixture at 90° C. for 2 h, the solution was concentrated in vacuo. To this acid chloride intermediate was added methylene chloride (5 mL) followed by ethyl anthranilate (412 mg, 2.5 mmol), and the reaction mixture was allowed to stir at room temperature for 5 h. Upon reaction completion, the mixture was washed with a solution of saturated sodium bicarbonate, brine, and dried over Na₂SO₄ and concentrated in vacuo. To the naphthol thiazole anthranilide ester (35 mg, 0.08 mmol) in THF (2 mL), MeOH (1 mL), and water (2 mL), was added aqueous 1M sodium hydroxide (36 mg, 0.9 mmol), and the reaction mixture was stirred at 50° C. for 12 h. Following reaction completion, the mixture was concentrated in vacuo, followed by the addition of water (5 mL), and the aqueous layer was acidified with concentrated HCl to pH 2. The acidic solution was extracted with ethyl acetate, and the organic layer was separated, dried over Na₂SO₄, and filtered. The filtrate was concentrated in vacuo and purified by reverse phase HPLC (Gilson) to give the desired compound. ¹H NMR (DMSO-d₆, 500 MHz) δ 12.1 (s, 1H), 10.1 (s, 1H), 8.49 (m, 2H), 7.99 (m, 3H), 7.81 (m, 1H), 7.66 (m, 1H), 7.23 (m, 1H), 7.17 (m, 1H); LCMS m/z 391 (M+1).

### Synthesis of DP Antagonist Compounds

Numerous DP receptor antagonist compounds have been published and are useful and included in the methods of the present invention. For example, DP receptor antagonists can be obtained in accordance with WO01/79169 published on Oct. 25, 2001, EP 1305286 published on May 2, 2003, WO02/094830 published on Nov. 28, 2002 and WO03/062200 published on Jul. 31, 2003. Compound AB can be synthesized in accordance with the description set forth in WO01/66520A1 published on Sep. 13, 2001; Compound AC can be synthesized in accordance with the description set forth in WO03/022814A1 published on Mar. 20, 2003, and Compounds AD and AE can be synthesized in accordance with the description set forth in WO03/078409 published on Sep. 25, 2003.

The synthesis of the remaining DP antagonist compounds disclosed herein can be undertaken using the description provided in WO2004/103370 published on Dec. 2, 2004.

### Biological Assays

The activity of the compounds of the present invention regarding niacin receptor affinity and function can be evaluated using the following assays:

### ³H-Niacin Binding Assay:

1. Membrane: Membrane preps are stored in liquid nitrogen in:
20 mM HEPES, pH 7.4
0.1 mM EDTA

Thaw receptor membranes quickly and place on ice. Resuspend by pipetting up and down vigorously, pool all tubes, and mix well. Use clean human at 15 m/well, clean mouse at 10 ug/well, dirty preps at 30 ug/well.
1a. (human): Dilute in Binding Buffer.
1b. (human+4% serum): Add 5.7% of 100% human serum stock (stored at −20° C.) for a final concentration of 4%. Dilute in Binding Buffer.
1c. (mouse): Dilute in Binding Buffer.
   2. Wash buffer and dilution buffer: Make 10 liters of ice-cold Binding Buffer:
20 mM HEPES, pH 7.4
1 mM MgCl₂
0.01% CHAPS (w/v)
use molecular grade or ddH₂O water
   3. [5,6-³H]-nicotinic acid: American Radiolabeled Chemicals, Inc. (cat #ART-689). Stock is ˜50 Ci/mmol, 1 mCi/ml, 1 ml total in ethanol→20 μM

Make an intermediate ³H-niacin working solution containing 7.5% EtOH and 0.25 μM tracer. 40 μL of this will be diluted into 200 μL total in each well→1.5% EtOH, 50 nM tracer final.

### 4. Unlabeled Nicotinic Acid:

Make 100 mM, 10 mM, and 80 μM stocks; store at −20° C. Dilute in DMSO.

### 5. Preparing Plates:

1) Aliquot manually into plates. All compounds are tested in duplicate. 10 mM unlabeled nicotinic acid must be included as a sample compound in each experiment.
2) Dilute the 10 mM compounds across the plate in 1:5 dilutions (8 μl:40 μl).
3) Add 195 μL binding buffer to all wells of Intermediate Plates to create working solutions (250 μM→0). There will be one Intermediate Plate for each Drug Plate.
4) Transfer 5 μL from Drug Plate to the Intermediate Plate. Mix 4-5 times.

### 6. Procedure:

1) Add 140 μL of appropriate diluted 19CD membrane to every well. There will be three plates for each drug plate: one human, one human+serum, one mouse.
2) Add 20 μL of compound from the appropriate intermediate plate
3) Add 40 μL of 0.25 μM ³H-nicotinic acid to all wells.
4) Seal plates, cover with aluminum foil, and shake at RT for 3-4 hours, speed 2, titer plate shaker.
5) Filter and wash with 8×200 μL ice-cold binding buffer. Be sure to rinse the apparatus with >1 liter of water after last plate.
6) Air dry overnight in hood (prop plate up so that air can flow through).
7) Seal the back of the plate
8) Add 40 μL Microscint-20 to each well.
9) Seal tops with sealer.
10) Count in Packard Topcount scintillation counter.
11) Upload data to calculation program, and also plot raw counts in Prism, determining that the graphs generated, and the IC₅₀ values agree.

The compounds of the invention generally have an IC₅₀ in the ³H-nicotinic acid competition binding assay within the range of about 10 nM to about 25 μM.

³⁵S-GTPγS binding assay:

Membranes prepared from Chinese Hamster Ovary (CHO)-K1 cells stably expressing the niacin receptor or vector control (7 μg/assay) were diluted in assay buffer (100 mM HEPES, 100 mM NaCl and 10 mM MgCl₂, pH 7.4) in Wallac Scintistrip plates and pre-incubated with test compounds diluted in assay buffer containing 40 μM GDP (final [GDP] was 10 μM) for ˜10 minutes before addition of ³⁵S-GTPγS to 0.3 nM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.

### Membrane Preparation

### Materials:

CHO-K1 cell culture medium: F-12 Kaighn's Modified Cell Culture Medium with 10% FBS, 2 mM L-Glutamine, 1 mM Sodium Pyruvate and 400 μ g̅/ml G418

### Membrane Scrape Buffer:

20 mM HEPES
10 mM EDTA, pH 7.4

### Membrane Wash Buffer:

20 mM HEPES
0.1 mM EDTA, pH 7.4
   Protease Inhibitor Cocktail: P-8340, (Sigma, St. Louis, Mo.)

### Procedure:

(Keep everything on ice throughout prep; buffers and plates of cells)
Aspirate cell culture media off the 15 cm² plates, rinse with 5 mL cold PBS and aspirate.
Add 5 ml Membrane Scrape Buffer and scrape cells. Transfer scrape into 50 mL centrifuge tube. Add 50 uL Protease Inhibitor Cocktail.
Spin at 20,000 rpm for 17 minutes at 4° C.
Aspirate off the supernatant and resuspend pellet in 30 mL Membrane Wash Buffer. Add 50 μL Protease Inhibitor Cocktail.
Spin at 20,000 rpm for 17 minutes at 4° C.
Aspirate the supernatant off the membrane pellet. The pellet may be frozen at −80° C. for later use or it can be used immediately.

### Assay Materials:

Guanosine 5′-diphosphate sodium salt (GDP, Sigma-Aldrich Catalog #87127)
Guanosine 5′-[γ³⁵S]thiotriphosphate, triethylammonium salt ([³⁵S]GTPγS, Amersham Biosciences Catalog #SJ1320, ˜100 Ci/mmol)
96 well Scintiplates (Perkin-Elmer #1450-501)

### Binding Buffer:

20 mM HEPES, pH 7.4
100 mM NaCl
10 mM MgCl₂
   GDP Buffer: binding buffer plus GDP, ranging from 0.4 to 40 μM, make fresh before assay

### Procedure:

(total assay volume=100μ well)
25 μL GDP buffer with or without compounds (final GDP 10 μM—so use 40 μM stock)
50 μL membrane in binding buffer (0.4 mg protein/mL)
25 μL [³⁵S]GTPγS in binding buffer. This is made by adding 5 μl [³⁵S]GTPγS stock into 10 mL binding buffer (This buffer has no GDP)
   Thaw compound plates to be screened (daughter plates with 5 μL compound @ 2 mM in 100% DMSO)
   Dilute the 2 mM compounds 1:50 with 245 μL GDP buffer to 40 μM in 2% DMSO. (Note: the concentration of GDP in the GDP buffer depends on the receptor and should be optimized to obtain maximal signal to noise; 40 μM).
   Thaw frozen membrane pellet on ice. (Note: they are really membranes at this point, the cells were broken in the hypotonic buffer without any salt during the membrane prep step, and most cellular proteins were washed away)
   Homogenize membranes briefly (few seconds—don't allow the membranes to warm up, so keep on ice between bursts of homogenization) until in suspension using a POLYTRON PT3100 (probe PT-DA 3007/2 at setting of 7000 rpm). Determine the membrane protein concentration by Bradford assay. Dilute membrane to a protein concentrations of 0.40 mg/ml in Binding Buffer. (Note: the final assay concentration is 20 μg/well).
   Add 25 μL compounds in GDP buffer per well to Scintiplate.
   Add 50 μL of membranes per well to Scintiplate.
   Pre-incubate for 5-10 minutes at room temperature. (cover plates with foil since compounds may be light sensitive)
   Add 25 μL of diluted [³⁵S]GTPγS. Incubate on shaker (Lab-Line model #1314, shake at setting of 4) for 60 minutes at room temperature. Cover the plates with foil since some compounds might be light sensitive.
   Assay is stopped by spinning plates sealed with plate covers at 2500 rpm for 20 minutes at 22° C.
   Read on TopCount NXT scintillation counter—35S protocol.

The compounds used in the invention generally have an EC₅₀ in the functional in vitro GTPγS binding assay within the range of about less than 1 μM to as high as about 100 μM.

### Flushing Via Laser Doppler

Male C57B16 mice (˜25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10^{th} image via injection into the peritoneal space. Images 1-10 are considered the animal's baseline and data is normalized to an average of the baseline mean intensities.

Materials and Methods—Laser Doppler Pirimed PimII; Niacin (Sigma); Nembutal (Abbott labs).

Compounds of this invention did not display flushing in this assay at doses as high as 100 mg/kg.

All patents, patent applications and publications that are cited herein are hereby incorporated by reference in their entirety. While certain preferred embodiments have been described herein in detail, numerous alternative embodiments are seen as falling within the scope of the invention.

1. A method of treating atherosclerosis, according to claim 19, in a mammalian patient in need of such treatment, comprising administering to the patient an anti-atherosclerotic effective amount of a compound represented by formula I: 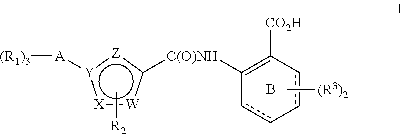
or a pharmaceutically acceptable salt or solvate thereof, wherein:
1-3 of W, X and Z are heteroatoms, and the remaining variable is a carbon atom; Y represents a carbon or nitrogen atom; 0-1 of W, X and Z represent an oxygen or sulfur atom, and the remainder of W, X and Z represent carbon or nitrogen atoms;
A represents a 9-10 membered aryl, an 8-10 membered heteroaryl or a partially aromatic heterocyclic group, said heteroaryl and partially aromatic heterocyclic group containing at least one heteroatom selected from O, S, S(O), S(O)₂ and N, and optionally containing 1 other heteroatom selected from O and S, and optionally containing 1-3 additional N atoms, with up to 5 heteroatoms being present;
each R1 represents H or is independently selected from the group consisting of:
a) OH, halo, CO2Ra, C(O)NRbRc, NRbRc, CN or S(O)pRd; and
b) C1-10alkyl, C2-10alkenyl, OC1-10alkyl or OC3-10alkenyl, said groups being optionally substituted with: (1) 1-5 halo groups up to a perhaloalkyl group; (2) 1 oxo group; (3) 1-2 OH groups; (4) 1 phenyl ring, which is optionally substituted as follows: 1-5 halo groups up to perhalo, 1-3 C1-10alkyl or alkoxy groups, each being further optionally substituted with 1-5 halo up to perhalo;
R² represents H or is selected from the group consisting of: C₁₋₃alkyl or C₂₋₃alkenyl, said alkyl and alkenyl group being optionally substituted with 1-3 halo atoms, and 1-2 OH, C₁₋₃alkoxy or haloC₁₋₃alkoxy groups;
R^{a} is H or C₁₋₄alkyl, optionally substituted with phenyl, OH, OC₁₋₆alkyl, CO₂H, CO₂C₁₋₆alkyl and 1-3 halo atoms;
R^{b} is H or C₁₋₄alkyl optionally substituted with 1-3 halo atoms and 1 phenyl, OH, and OC₁₋₆alkyl group;
R^{c} is H or is independently selected from: (a) C₁₋₄alkyl, and (b) Aryl or Ar—C₁₋₄alkyl, each optionally substituted with 1-3 halo atoms and 1-3 members selected from the group consisting of: CN, OH, C₁₋₃alkyl and OC₁₋₃alkyl, said alkyl and alkoxy being further optionally substituted with 1-3 halo atoms;
R^{d} is selected from: (a) C₁₋₄alkyl, (b) Aryl or Ar—C₁₋₄alkyl, each optionally substituted with 1-3 halo atoms and 1-3 members selected from the group consisting of: CN, OH, C₁₋₃alkyl and OC₁₋₃ alkyl, said alkyl and alkoxy being further optionally substituted with 1-3 halo atoms;
p is an integer selected from 0, 1 and 2;
and the dotted lines in ring B represent bonds which are either both present or both absent, such that when the bonds are present, ring B is a phenyl ring, and each R³ represents H, halo, methyl or methyl substituted with 1-3 halo atoms;
and when the optional bonds are absent, ring B is a cyclohexene ring and each R³ represents H, halo, C₁₋₃alkyl, Aryl and HAR,
said C₁₋₃alkyl, Aryl and HAR being optionally substituted with 1-3 groups, 0-3 of which are halo, and 0-1 of which are selected from the group consisting of: OH, NH₂, NHC₁₋₃alkyl, N(C₁₋₃alkyl)₂, CN, C₁₋₃alkyl, C₁₋₃alkoxy, haloC₁₋₃alkyl, haloC₁₋₃alkoxy, and Hetcy groups.

2. A method of treating atherosclerosis in accordance with claim 1 wherein:
A represents a member selected from the group consisting of: naphthyl, quinolinyl, isoquinolinyl, quinoxalinyl, benzodioxanyl, benzodioxolanyl, benzodihydrofuranyl and benzothiazolyl.

3. A method of treating atherosclerosis in accordance with claim 1 wherein: Z represents a sulfur atom and W, X and Y represent carbon atoms.

4. A method of treating atherosclerosis in accordance with claim 1 wherein: W represents a sulfur atom and Z, X and Y represent carbon atoms.

5. A method of treating atherosclerosis in accordance with claim 1 wherein: W and Z represent carbon atoms, and X and Y represent nitrogen atoms.

6. A method of treating atherosclerosis in accordance with claim 1 wherein W and X represent carbon atoms, and Y and Z represent nitrogen atoms.

7. A method of treating atherosclerosis in accordance with claim 1 wherein: W and Y represent carbon atoms, X represents a sulfur atom and Z represents a nitrogen atom.

8. A method of treating atherosclerosis in accordance with claim 1 wherein W and Y represent carbon atoms, X represents a nitrogen atom and Z represents a sulfur atom.

9. A method of treating atherosclerosis in accordance with claim 1 wherein: 1-2 R¹ groups represent H and the remaining R¹ groups are selected from the group consisting of: H, halo, OH, NH₂ and methoxy.

10. A method of treating atherosclerosis in accordance with claim 1 wherein: R² represents H or methyl.

11. A method of treating atherosclerosis in accordance with claim 1 wherein:
R³ represents H.

12. A method of treating atherosclerosis in accordance with claim 1 wherein:
A represents a member selected from the group consisting of: naphthyl, quinolinyl, isoquinolinyl, quinoxalinyl, benzodioxanyl, benzodioxolanyl, benzodihydrofuranyl and benzothiazolyl;
Z represents a sulfur atom and W, X and Y represent carbon atoms, or
W represents a sulfur atom and Z, X and Y represent carbon atoms, or
W and Z represent carbon atoms, and X and Y represent nitrogen atoms, or
W and Y represent carbon atoms, X represents a sulfur atom and Z represents a nitrogen atom, or
W and X represent carbon atoms and Y and Z represent nitrogen atoms, or
W and Y represent carbon atoms, X represents a nitrogen atom and Z represents a sulfur atom;
1-2 R¹ groups represent H and the remaining R¹ groups are selected from the group consisting of: H, halo, OH, NH₂ and methoxy;
R² represents H or methyl, and each R³ represents H.

13. A method in accordance with claim 1 wherein the compound administered is selected from the following table:
TABLE 1 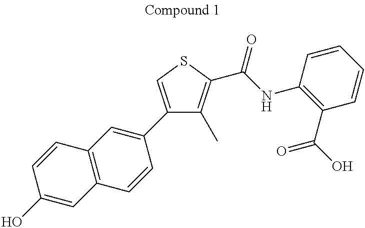 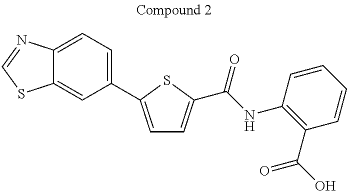 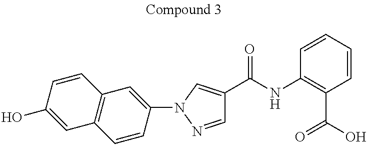 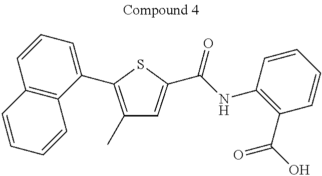 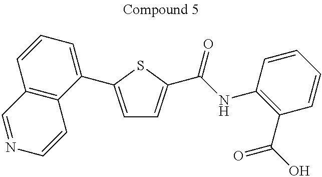 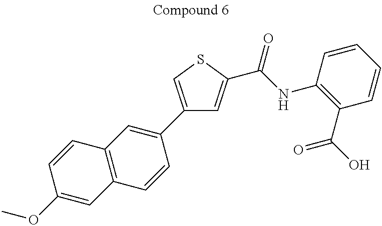 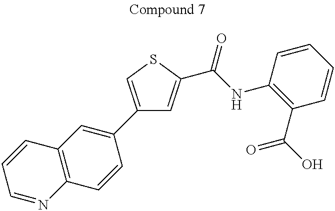 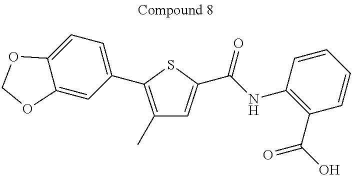 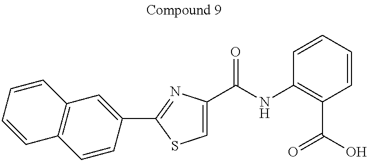 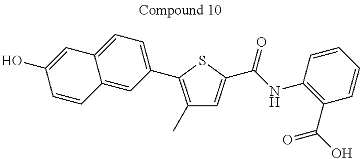 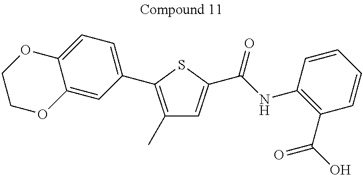 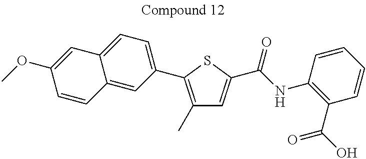 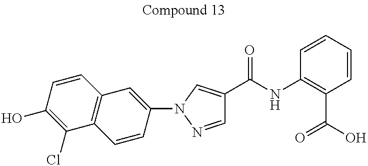 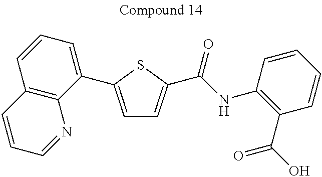 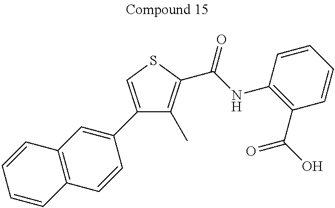 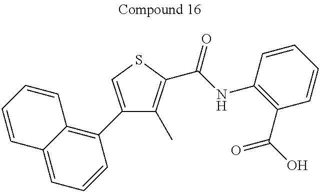 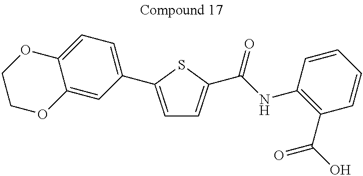 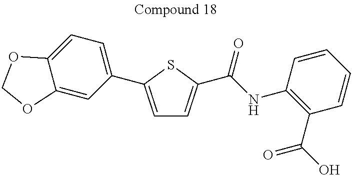 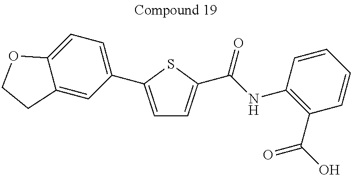 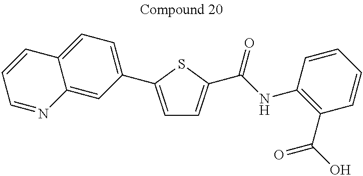 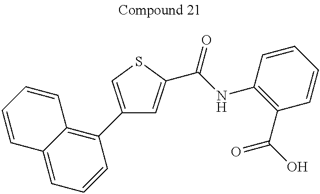 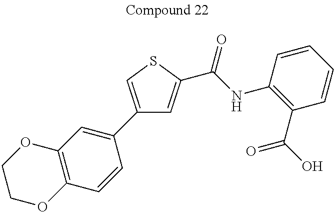 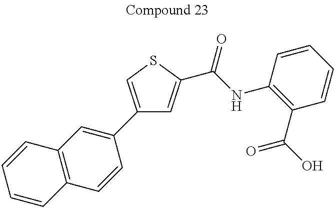 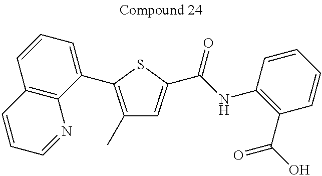 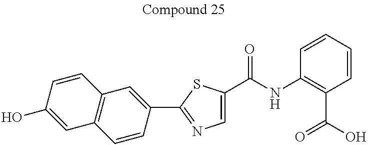
or a pharmaceutically acceptable salt or solvate thereof.

14. A compound selected from the group consisting of:
TABLE 1 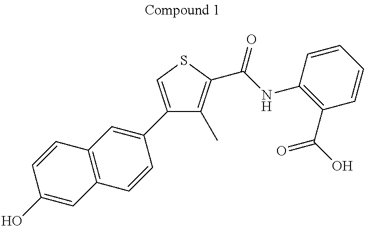 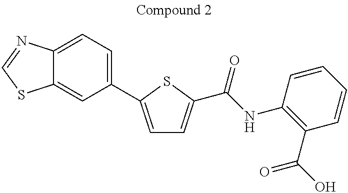 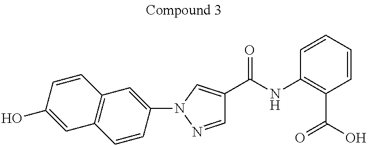 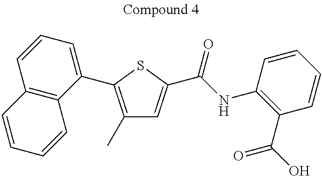 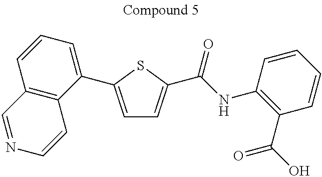 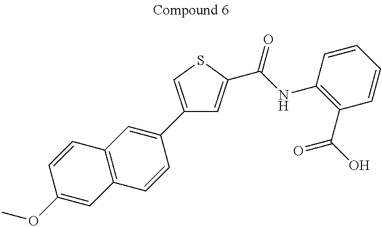 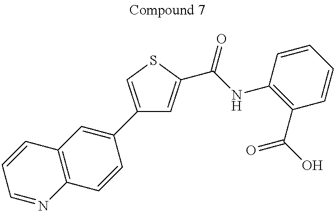 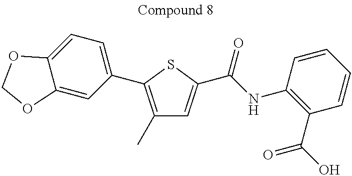 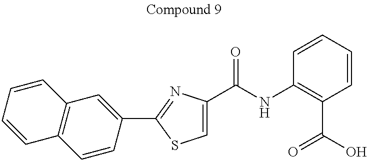 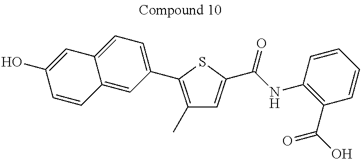 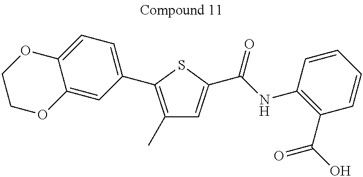 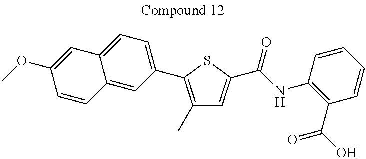 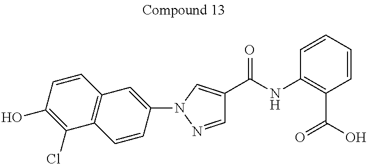 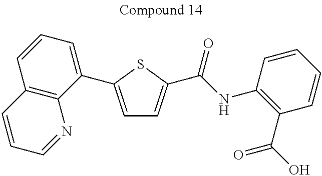 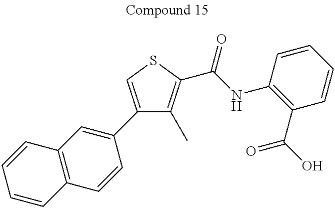 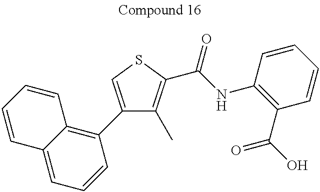 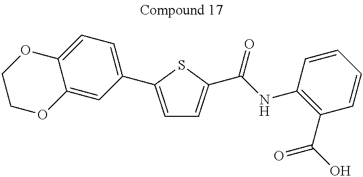 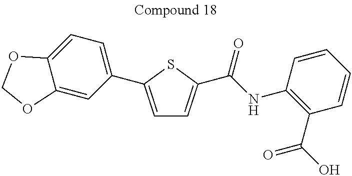 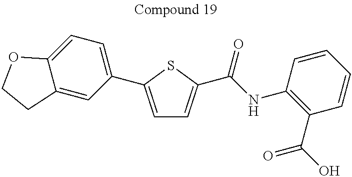 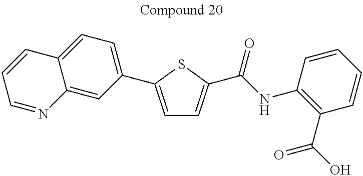 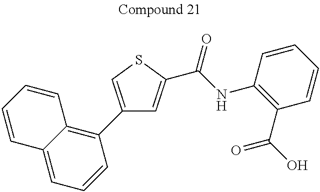 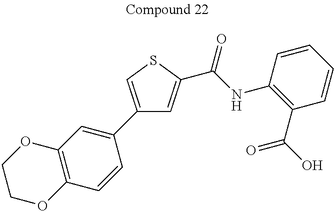 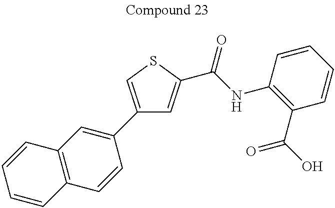 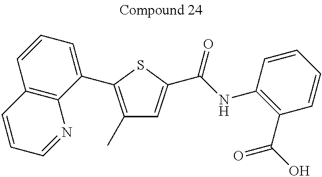 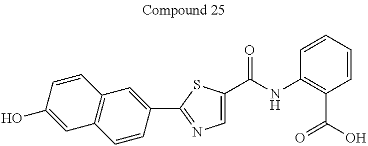
and the pharmaceutically acceptable salts and solvates thereof.

15. A pharmaceutical composition comprised of a compound in accordance with claim 11 in combination with a pharmaceutically acceptable carrier.

16. (canceled)

17. (canceled)

18. (canceled)

19. A method of treating atherosclerosis, dyslipidemia, diabetes, metabolic syndrome or a related condition in a human patient in need of such treatment, comprising administering to the patient a compound of the formula I: 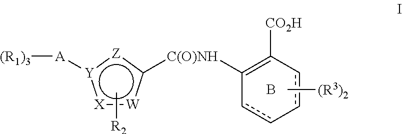
or a pharmaceutically acceptable salt or solvate thereof, wherein:
1-3 of W, X and Z are heteroatoms, and the remaining variable is a carbon atom; Y represents a carbon or nitrogen atom; 0-1 of W, X and Z represent an oxygen or sulfur atom, and the remainder of W, X and Z represent carbon or nitrogen atoms;
A represents a 9-10 membered aryl, an 8-10 membered heteroaryl or a partially aromatic heterocyclic group, said heteroaryl and partially aromatic heterocyclic group containing at least one heteroatom selected from O, S, S(O), S(O)₂ and N, and optionally containing 1 other heteroatom selected from O and S, and optionally containing 1-3 additional N atoms, with up to 5 heteroatoms being present;
each R¹ represents H or is independently selected from the group consisting of:
a) OH, halo, CO2Ra, C(O)NRbRc, NRbRc, CN or S(O)pRd; and
b) C1-10alkyl, C2-10alkenyl, OC1-10alkyl or OC3-10alkenyl, said groups being optionally substituted with: (1) 1-5 halo groups up to a perhaloalkyl group; (2) 1 oxo group; (3) 1-2 OH groups; (4) 1 phenyl ring, which is optionally substituted as follows: 1-5 halo groups up to perhalo, 1-3 C1-10alkyl or alkoxy groups, each being further optionally substituted with 1-5 halo up to perhalo;
R² represents H or is selected from the group consisting of: C₁₋₃alkyl or C₂₋₃alkenyl, said alkyl and alkenyl group being optionally substituted with 1-3 halo atoms, and 1-2 OH, C₁₋₃alkoxy or haloC₁₋₃alkoxy groups;
R^{a} is H or C₁₋₄alkyl, optionally substituted with phenyl, OH, OC₁₋₆alkyl, CO₂H, CO₂C₁₋₆alkyl and 1-3 halo atoms;
R^{b} is H or C₁₋₄alkyl optionally substituted with 1-3 halo atoms and 1 phenyl, OH, and OC₁₋₆alkyl group;
R^{c} is H or is independently selected from: (a) C₁₋₄alkyl, and (b) Aryl or Ar—C₁₋₄alkyl, each optionally substituted with 1-3 halo atoms and 1-3 members selected from the group consisting of: CN, OH, C₁₋₃alkyl and OC₁₋₃ alkyl, said alkyl and alkoxy being further optionally substituted with 1-3 halo atoms;
R^{d} is selected from: (a) C₁₋₄alkyl, (b) Aryl or Ar—C₁₋₄alkyl, each optionally substituted with 1-3 halo atoms and 1-3 members selected from the group consisting of: CN, OH, C₁₋₃alkyl and OC₁₋₃ alkyl, said alkyl and alkoxy being further optionally substituted with 1-3 halo atoms;
p is an integer selected from 0, 1 and 2;
and the dotted lines in ring B represent bonds which are either both present or both absent, such that when the bonds are present, ring B is a phenyl ring, and each R³ represents H, halo, methyl or methyl substituted with 1-3 halo atoms;
and when the optional bonds are absent, ring B is a cyclohexene ring and each R³ represents H, halo, C₁₋₃alkyl, Aryl and HAR,
said C₁₋₃alkyl, Aryl and HAR being optionally substituted with 1-3 groups, 0-3 of which are halo, and 0-1 of which are selected from the group consisting of: OH, NH₂, NHC₁₋₃alkyl, N(C₁₋₃alkyl)₂, CN, C₁₋₃alkyl, C₁₋₃alkoxy, haloC₁₋₃alkyl, haloC₁₋₃alkoxy, and Hetcy groups.
and a DP receptor antagonist, said combination being administered in an amount that is effective to treat atherosclerosis, dyslipidemia, diabetes or a related condition in the absence of substantial flushing.

20. A method of treating atherosclerosis, dyslipidemias, diabetes or a related condition, in accordance with claim 19, in a human patient in need of such treatment, comprising administering to the patient a compound of Formula I and a DP receptor antagonist selected from the group consisting of compounds A through AJ: 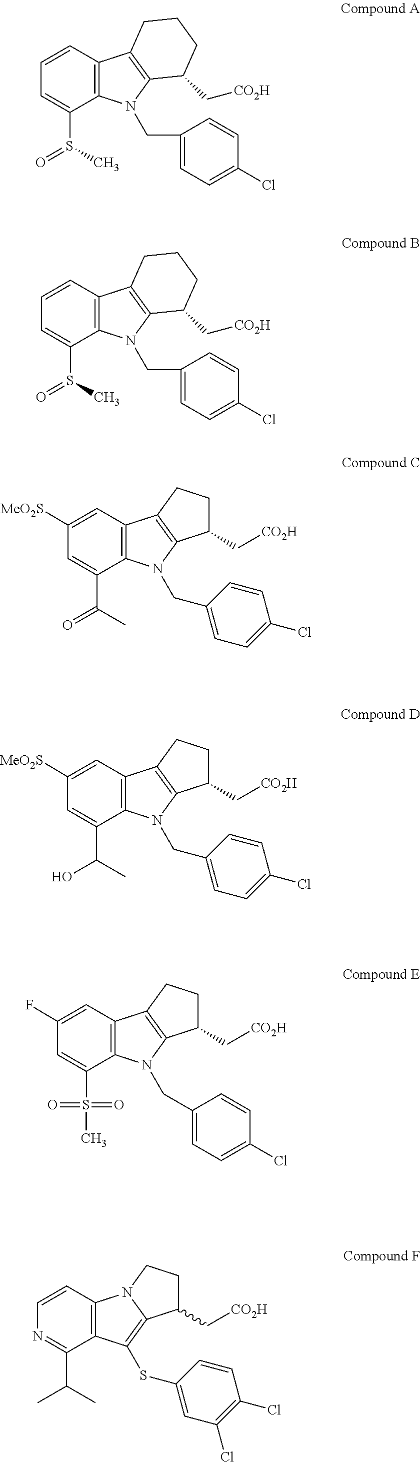 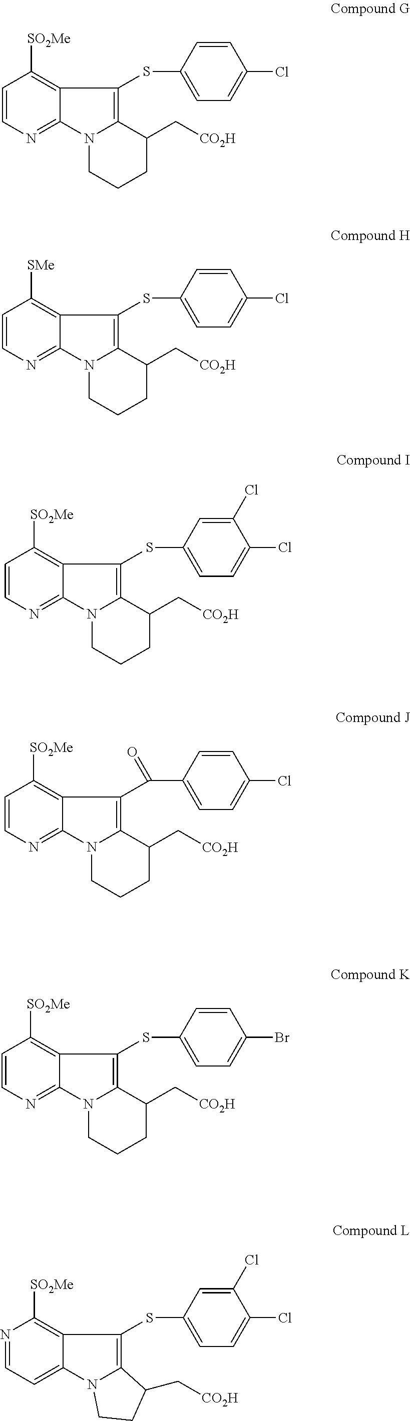 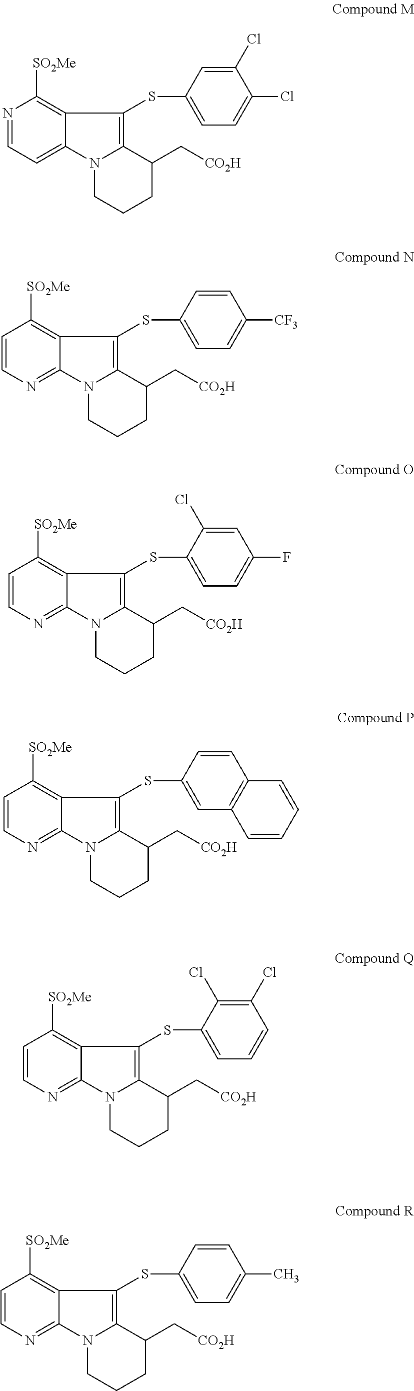 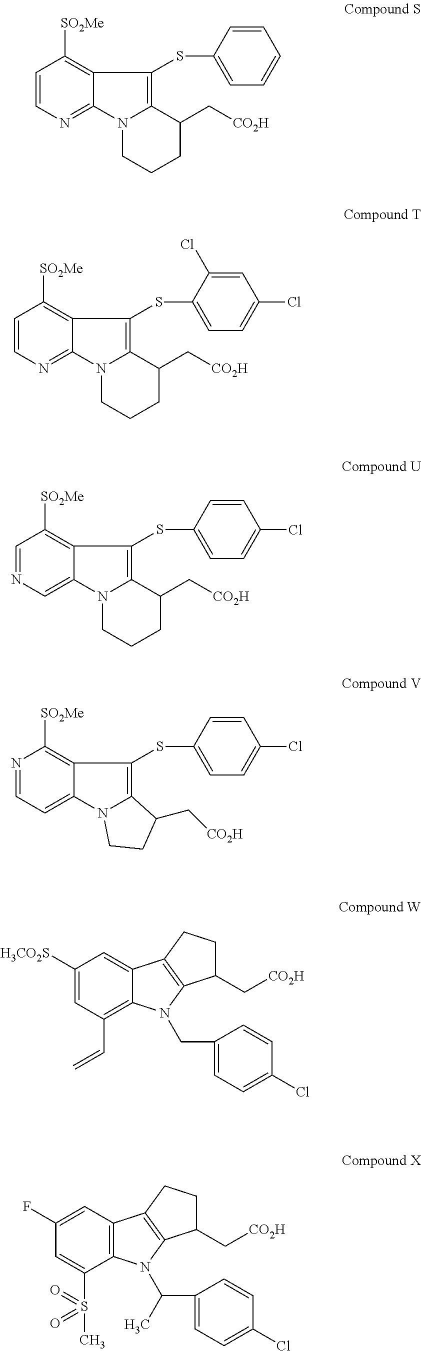 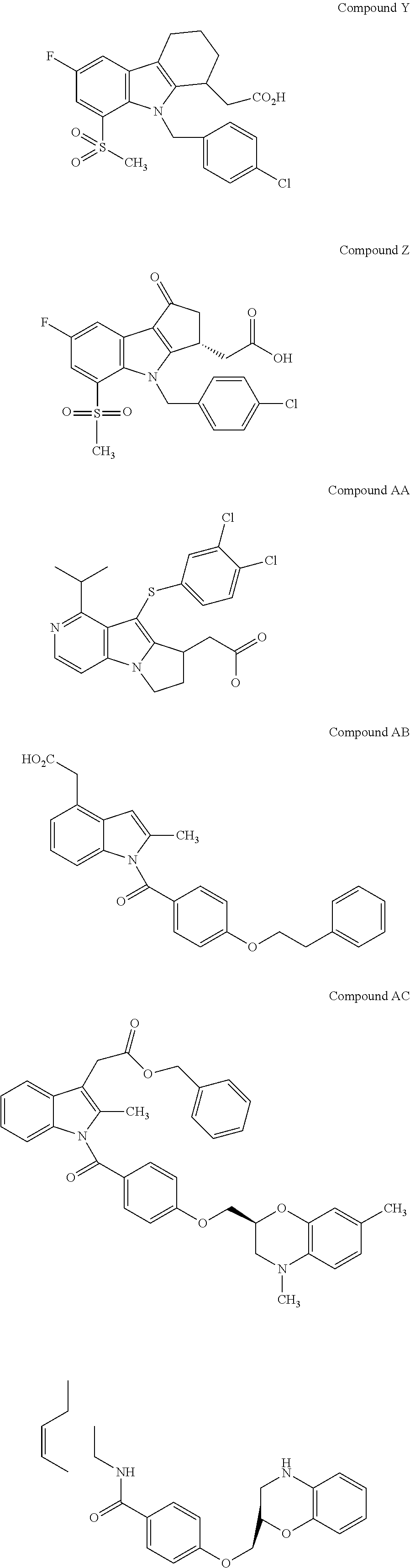 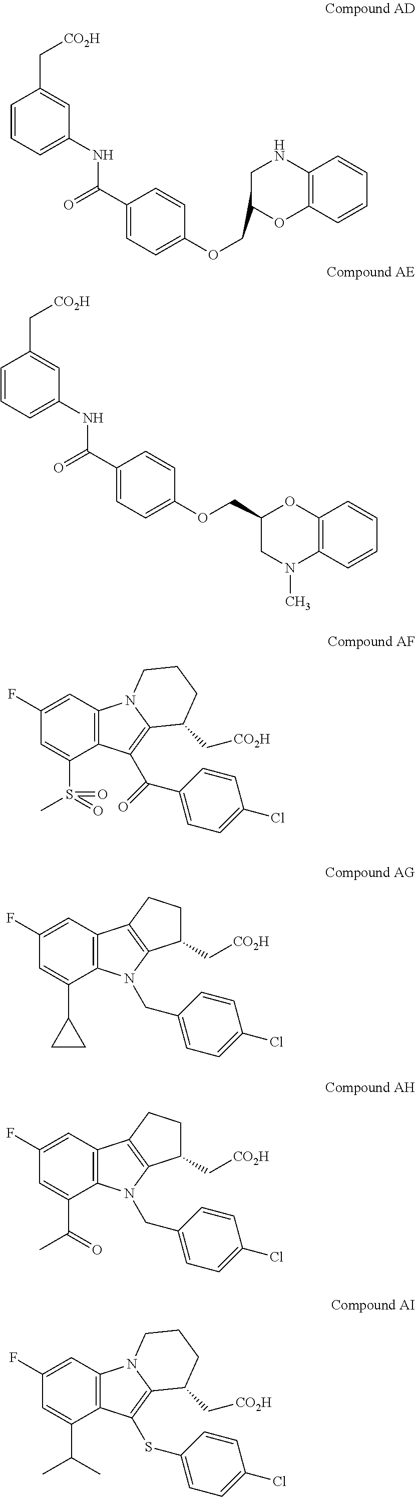 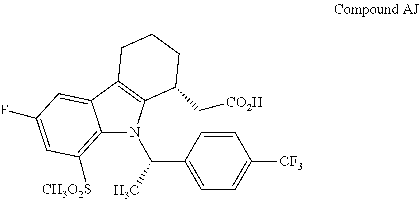
or a pharmaceutically acceptable salt or solvate thereof.
